(19) United States
(12) Patent Application Publication
(10) Pub. No.: US 2024/0310348 A1
(43) Pub. Date: Sep. 19, 2024

(54) INDICATOR DEVICE AND SYSTEM AND USES THEREOF FOR DETECTING FOOD SPOILAGE

(71) Applicant: BIO TIP LTD, Tel Aviv-Yafo (IL)

(72) Inventors: Zur GRANEVITZE, Mazkeret Batya (IL); Ben-Zion MAGNES, Meitar (IL)

(21) Appl. No.: 18/674,828

(22) Filed: May 25, 2024

Related U.S. Application Data
(63) Continuation of application No. PCT/IL2022/051337, filed on Dec. 15, 2022.
(60) Provisional application No. 63/290,312, filed on Dec. 16, 2021.

(51) Int. Cl. G01N 33/02; G01N 31/22

(52) U.S. Cl. CPC G01N 33/02; G01N 31/221

(57) ABSTRACT

Provided herein are devices for determining freshness of food products and for detecting food spoilage, which include a substrate and an indicator composition. Further provided are systems including the same and uses thereof.

### CROSS REFERENCE TO RELATED APPLICATIONS

This application is a Bypass Continuation of PCT Patent Application No. PCT/IL2022/051337 having International filing date of Dec. 15, 2022, which claims the benefit of priority of U.S. Provisional Patent Application No. 63/290,312, filed Dec. 16, 2021, the contents of which are all incorporated herein by reference in their entirety.

### FIELD OF THE INVENTION

Provided herein are devices for determining freshness of food products, and for detecting food spoilage, systems including the same and methods of use thereof.

### BACKGROUND

Spoilage of fresh food products with time is an on-going challenge for the consumers and the food industry. Labeling of ‘Expiration Date’, ‘Use Before’, ‘Best Before’ dates are often found non-reliable, as in many instances they do not reflect the actual quality of a food item (freshness). It is well known that during the numerous marketing and consumption stages (packaging, transportation, distribution, storage, home usage etc.) the conditions required for maintaining food freshness are not always adequate, which may lead to spoilage prior to the expected expiration date.

Moreover, expiration dates are calculated for an entire food category for all batches (and not per food package or even per batch) and take a significant caution factor. Thus, by definition they are not accurate and cause waste of over about 10% of total food production which is done by the manufacturers, retailers and end consumers.

There is therefore an unmet need for an indicator device and system capable of providing a continuous real time indication of food freshness or spoilage or pathogen presence per food package, which is cost effective and easy to implement on or with existing food packages.

### SUMMARY

Disclosed herein is an indicator device configured to provide real-time indication of food freshness, food spoilage and/or pathogen presence. Advantageously, the device is configured to enable unidirectional contact between the food products, or reactants secreted therefrom and an indicator agent (indicator composition), while preventing/avoiding or allowing only minor transition of the indicator agent to the food product. The unidirectional contact could be instant, namely, a one-time contact, or continuous. Nevertheless, the indication remains informative and reliable at any one of the aforementioned configurations.

According to some embodiments, the indicator device may be in continuous contact with the food product or in a continuous contact with a portion thereof, following a brief, transient, passage of the food product, a portion thereof, or components secreted therefrom, into the indicator device. Accordingly, the contact between the food product and the indicator composition/device may be automatic, or may be enabled manually, namely, following opening of a passage between the indicator device and the food product. According to some embodiments, the contact may be direct or in-direct. In some embodiments, the contact is fluid (gas or liquid) contact. In some embodiments, the device may be placed/associated directly with the food product and the contact between the food and the indicator portion of the device may be facilitated automatically/spontaneously.

In some embodiments, the indicator device is configured to be placed/positioned on a food container/package (permanently or transiently), and to provide indication regarding the food product therewithin, by enabling the transient, unidirectional passage of the food product, a portion thereof, or components secreted therefrom, into the indicator device.

In some embodiments, the indicator device is configured to be placed/positioned directly on a food (permanently or transiently), and to provide indication regarding the food product, by enabling transient, preferably unidirectional passage of components present on/with or secreted from the food product to the indicator device.

According to some embodiments, the indicator device or indicator composition may be placed within or in direct contact with the food and the ‘spoilage signal” (for example, color change reaction, spectroscopic indication, colorimetric indication, fluorimetry, electrochemical indication, phase transition (such as phase separation), sedimentation or gas evolution) may be conveyed/transmitted externally to the food surface or the food package. Such transmission of the internal spoilage signal (e.g., color change) may be done, for example, by mirror reflection, magnifying glass, electronic device, or any other suitable mean.

In some embodiments, the device indicator is configured to be placed/positioned/located on individual or a plurality of food containers, wherein the food product within the container may be any type of food, with any type of shape, form, or composition, including, for example, solid, semi solid, semi liquid, liquid, frozen, defrosted, and the like forms.

According to some embodiments, there is provided an indicator device for detecting food spoilage of a food product, the indicator device includes
a. a substrate; and
b. an indicator composition including at least one indicator, the indicator composition is associated with the substrate,
wherein said substrate is configured to be in contact with the food product, said indicator composition is configured to produce a detectable signal upon reacting with at least a portion of the food product, or of components secreted therefrom.

According to some embodiments, the substrate may include or be made of: textile fibers, plastic, nylon, woven fibers, woven fabrics, non-woven fabrics, artificial polymers, biodegradable polymers, cotton, wool, linen, oligo-sugars, gels, modified starch, metals, aluminum, paper, carton, wood, plant material, clay, adhesives, ointment, vax, oil, leather, carbon and graphite felt, carbon fibers, or any combination thereof.

According to some embodiments, the substrate may include or be made of: textile fibers, plastic, nylon, woven fibers, non-woven fibers, cotton, silk, wool, filter paper (such as, coffee filter), or any combination thereof. In some embodiments, the substrate may further include a coating layer, facilitating the placement/attachment to the food product or food packaging.

According to some embodiments, the substrate may further include one or more additional layers. In some embodiments, at least one of the layers is configured to at least partially prevent leakage of indicator composition to the food product.

According to some embodiments, the additional layer may include or be made of textile fibers, plastic, nylon, woven fibers, non-woven fibers, cotton, wool, silk, filter paper, cellulose-starch chitosan-PVA, cellulose-PVA cross linked film, or any combination thereof. In some embodiments, the substrate and/or the additional layer may be treated with various substances to enhance various and/or association with the indicator composition, including such substances as, but not limited to: non organic salts, organic cationic salts, proteins (for example, food approved), and the like, or any combination thereof.

According to some embodiments, the additional layer may be permanently or transiently associated with the substrate. In some embodiments, the additional layer may be stitched, adhered, glued and/or welded to the substrate.

According to some embodiments, the spoilage is a microbial related spoilage, including any type of microorganisms, including, for example, but not limited to: bacteria, fungi, mold, pathogens, and the like.

According to some embodiments, the at least one indicator may be selected from microbial indicators, azo dye, food colors, natural food dyes, Thiazine dye, redox indicators, pH indicators, oxygen indicator, solvatochromic dyes, nano biosensors, such as, gold nanoparticles, silver nanoparticles, quantum dots, magnetic nanoparticles, single and/or multiwall nanotubes, graphene and reduced graphene flakes, TiO2 nanoparticles, fluorescent polymeric nanoparticles, nanochips, nanowires, fiber-optic biosensors, or any combination thereof. Each possibility is a separate embodiment.

According to some embodiments, the at least one indicator may be provided in a concentration such that a change (such as, visual change) is detectable (for example, changes its color, spectroscopic, colorimetric change, fluorimetry change, electrochemical change), in response to spoilage of the food sample. According to some embodiments, the at least one indicator may be a pH indicator and wherein the indicator may change color upon a change of pH smaller than about 0.1 pH unit. According to some embodiments, the at least one indicator may be a pH indicator and wherein the indicator may change color upon a change of pH smaller than about 0.5 pH unit. According to some embodiments, the at least one indicator may be a bacterial indicator and may be devoid of quaternary ammonium salts and quaternary amine moieties. According to some embodiments, the at least one bacterial indicator may provide a detectable indication in the presence of a bacterial population above a pre-determined threshold. According to some embodiments, the at least one bacterial indicator may provide a detectable indication in the presence of volatile compounds secreted from the food product above a threshold, for example, due to bacterial population above a pre-determined threshold. For example, the indictor composition may include ninhydrin, phthalocyanines and/or azo dyes, which form colored complexes with volatile amines, which are products of bacterial decomposition of proteins (biogenic amines), such as putrescine, ammonia, urea and other.

According to some embodiments, the at least one indicator may be selected from bromothymol blue, cresol red, phenol red, methyl red, bromocresol blue, indigo carmine, carmoisine red, tartrazine, bromocresol green, methyl orange, brown HT, methyl blue, sunset yellow FCF, citrus red 2, amaranth, Ponceau 4R, quinoline yellow, carmoisine, patent blue V, green S, brilliant blue, indigotine, fast green FCF, methylene blue, erythosine, allura red AC, orange B, scarlet GN, sudan II, acid orange 20, annatto, caramel coloring, carmine, elderberry, lycopene, paprika, turmeric, tetrazolium salts, resazurin, ninhydrin, phthalocyanines, azo dye, or any combination thereof.

According to some embodiments, the indicator composition may include a plurality of indicators.

According to some embodiments, the indicator composition may be linked to the substrate. In some embodiments, the indicator composition is essentially irreversibly linked to the substrate. In some embodiments, the indicator composition may be reversibly linked to the substrate. In some embodiments, the indicator composition may be covalently linked to the substrate.

According to some embodiments, the substrate may be associated with a plurality of indicator compositions. According to some embodiments, the plurality of indicator compositions may be spatially distinct.

According to some embodiments, the plurality of indicator compositions may be different with respect of type of indicator and/or concentration of indicator.

According to some embodiments, the substrate may be in fluid (such as, gas or liquid) contact with the food sample. In some embodiments, the substrate may be configured to be in direct or in-direct contact with the food product

According to some embodiments, the device may be in the form of a patch, a sticker, a flag, a note, and the like.

According to some embodiments, the detectable signal may qualitative or quantitative. In some embodiments, the detectable signal is colorimetric, spectroscopic, fluorometry and/or electrochemical.

According to some embodiments, the indicator device may be or is included with a food package configured to at least partially hold or wrap the food.

According to some embodiments, there is provided a method for detecting food spoilage of a food product, the method includes the steps of: placing the indicator device as disclosed herein, in contact with a food product; allowing at least a food sample of said food product to contact said indicator; and determining whether said food product is spoiled upon detecting a detectable signal generated by said indicator, if/when the food sample is spoiled.

According to some embodiments, there is provided a system for detecting food spoilage of a food product, the system includes at least one food packaging configured to at least partially hold or wrap a food product; and an indicator device as disclosed herein, associated with the food packaging.

According to some embodiments, there is provided an indicator device configured to associate with a food package (capable of holding a food product) and indicate spoilage of a food comprised in the food package, the indicator device includes:
(a) a substrate comprising an indicator composition, the indicator composition includes at least one indicator; and
(b) at least one passage configured to enable a unidirectional passage of a food sample (from the food package) to the indicator device;
wherein the indicator composition is configured to produce a detectable signal upon reacting with the food sample, said signal is indicative of food spoilage.

According to some embodiments, the food sample may include portion of the food product and/or components secreted therefrom.

According to some embodiments, the indicator composition may be associated with the substrate.

According to some embodiments, the spoilage is a microbial related spoilage.

According to some embodiments, the at least one indicator may be selected from microbial indicators, azo dye, food colors, natural food dyes, Thiazine dye, redox indicators, pH indicators, oxygen indicators, solvatochromic dyes, or any combination thereof.

According to some embodiments, the substrate may include or be made of: textile fibers, plastic, nylon, woven fibers, woven fabrics. non-woven fabrics, artificial polymers, biodegradable polymers, cotton, wool, linen, oligo-sugars, gels, modified starch, metals, aluminum, paper (such as filter paper, for example, coffee filter), carton, wood, plant material, clay, adhesives, ointment, vax, oil, leather, silk, or any combination thereof. Each possibility is a separate embodiment.

According to some embodiments, the at least one indicator may be provided in a concentration such that a detectable signal is facilitated (for example, a change in color) in response to spoilage of the food sample. According to some embodiments, the at least one indicator may be a pH indicator and wherein the indicator may change color upon a change of pH smaller than about 0.1 pH unit. According to some embodiments, the at least one indicator may be a pH indicator and wherein the indicator may change color upon a change of pH smaller than about 0.5 pH unit. According to some embodiments, the at least one indicator may be a bacterial indicator and may be devoid of quaternary ammonium salts and quaternary amine moieties. According to some embodiments, the at least one bacterial indicator may provide a detectable indication in the presence of a bacterial population above a pre-determined threshold.

According to some embodiments, the at least one indicator may be selected from bromothymol blue, cresol red, phenol red, methyl red, bromocresol blue, indigo carmine, carmoisine red, tartrazine, bromocresol green, methyl orange, methyl blue, sunset yellow FCF, citrus red 2, amaranth, Ponceau 4R, quinoline yellow, carmoisine, patent blue V, green S, brilliant blue, indigotine, fast green FCF, erythosine, allura red AC, orange B, scarlet GN, sudan II, acid orange 20, annatto, caramel coloring, carmine, elderberry, lycopene, paprika, turmeric, tetrazolium salts, resazurin, ninhydrin, phthalocyanines, azo dye, or any combination thereof.

According to some embodiments, the indicator composition may include a plurality of indicators.

According to some embodiments, the indicator composition may be linked to the substrate. In some embodiments, the indicator composition is essentially irreversibly linked to the substrate. In some embodiments, the indicator composition may be reversibly linked to the substrate. In some embodiments, the indicator compression may be covalently linked to the substrate.

According to some embodiments, the at least one passage may be a closed passage configured to be transiently opened when required.

According to some embodiments, the at least one passage may include at least one capillary, membrane, displacement element, or any combination thereof. According to some embodiments, the displacement may be rotation or press.

According to some embodiments, the least one passage may to configured to be opened one or more times.

According to some embodiments, the indicator composition may be linked to the substrate. In some embodiments, the indicator composition is essentially irreversibly linked to the substrate. In some embodiments, the indicator composition may be reversibly linked to the substrate. In some embodiments, the indicator compression may be covalently linked to the substrate.

According to some embodiments, the substrate may be associated with a plurality of indicator compositions. According to some embodiments, the plurality of indicator compositions may be spatially distinct.

According to some embodiments, the plurality of indicator compositions may be different with respect of type of indicator and/or concentration of indicator.

According to some embodiments, the substrate may be in fluid (such as, gas or liquid) contact with the food sample. In some embodiments, the substrate may be configured to be in direct or in-direct contact with the food product

According to some embodiments, the substrate may be in fluid (such as, gas or liquid) contact with the food sample. In some embodiments, the substrate may be configured to be in direct or in-direct contact with the food product.

According to some embodiments, the detectable signal may qualitative or quantitative. In some embodiments, the detectable signal may be colorimetric.

According to some embodiments, there is provided a method of detecting food spoilage of a food product comprised in a food package, the method includes the steps of associating the indicator device to be in contact with the food packaged; transiently allowing unidirectional food sample passage from the food package towards the indicator device, by opening the closed passage, thereby allowing the food sample to contact the indicator composition and to react therewith; and determining whether said food product is spoiled upon detecting the detectable signal.

According to some embodiments, there is provided a system for detecting food spoilage of a food product, the system includes:
at least one food packaging configured to hold a food product; and an indicator device as disclosed herein (which includes at least one passage), associated with the food packaging.

According to some embodiments, the food product may be selected from fluid food product, liquid food product, soft food product, semi-hard food product, hard food product and aqueous food product.

According to some embodiments, the food packaging may be selected from a container, a vial, a box, a tray, a can and a palette, a bag, wrap, nylon wrap, saran wrap, smart package, or any combinations thereof.

According to some embodiments, the indicator device may be integrally formed with the food packaging. According to some embodiments, the indicator device may be permanently associated with the food packaging. According to some embodiments, the indicator device may be transiently associated with the food packaging. According to some embodiments, the indicator device may be associated with an external region of the food packaging.

Other objects, features and advantages of the present invention will become clear from the following description, examples and drawings.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIGS. 1A-1C show pictograms of four vials, each containing whole milk and a piece of wool dyed with 50 ppm Indigo Carmine solution (i.e., wool dyed with Indigo Carmine solution at concentration of 50 ppm) following incubation at room temperature (25-27° C.) at t=0 (FIG. 1A), after about 30 hours (FIG. 1B) and after about 48 hours (FIG. 1C);

FIGS. 2A and 2B present fresh samples of milk, each containing a piece of wool dyed with Indigo Carmine solution at a concentration of 60 ppm before and after 36-40 hours incubation in room temperature (25-27° C.), respectively.

FIGS. 3A and 3B present vials containing chicken breast or red meet wrapped within cotton fabric stained with neutral or basic cationic solution, respectively, at t=0.

FIGS. 3C and 3D present vials shown in FIGS. 3A and 3B, respectively, after being incubated in room temperature for 30-36 hours (26-28° C.);

FIGS. 4A and 4B present vials containing cottage cheese wrapped within cotton fabric stained with neutral cationic solution, at t=0 and t=28 hours, respectively.

FIGS. 5A-5C present cotton fabric stained with neutral cationic solution, at t=0, t=28−30 and t=48 hours, respectively, within vials containing soy milk at room temperature (26-28° C.);

FIGS. 6A-6B present pictograms of vials containing cotton fabric associated with indigo carmine indicator, incubated at room temperature (26-28° C.), in the presence of Cottage cheese at t=0 (FIG. 6A), and t=24 (FIG. 6B); Each pictogram shows vials containing only a food sample (cottage cheese), vials containing only cotton fabric associated with indigo carmine indicator and vials containing both cotton fabric associated with indigo carmine indicator and food sample.

FIGS. 6C-6D present pictograms of vials containing cotton fabric associated with indigo carmine indicator, incubated at room temperature (26-28° C.), in the presence of minced chicken breast at t=0 (FIG. 6C), and t=24 (FIG. 6D). Each pictogram shows vials containing only a food sample (minced chicken breast), vials containing only cotton fabric associated with indigo carmine indicator and vials containing both cotton fabric associated with indigo carmine indicator and food sample;

FIGS. 7A-7B present pictograms of fresh fish meat (salmon) having an indicator device associated with the packaging of the meat (the packaging in the form of Nylon wrap). Shown in FIG. 7A is fresh meat (t=0) and in FIG. 7B spoiled meat (after 24-36 hours in room temperature) is shown. The change in color of the indicator on the fresh meat (FIG. 7A) and on the spoiled meat (FIG. 7B) is visible;

FIGS. 8A-C show perspective illustrations of an indicator device, according to some embodiments. FIG. 8A—shows an illustration of an indictor device; FIG. 8B—shows pictograms and corresponding illustrations of layers/portions of an indictor device; FIG. 8C shows pictograms of indictor device, and layers thereof, while being associated with a food product;

FIG. 9 show perspective illustrations of an indicator device allowing unidirectional food sample passage, according to some embodiments.

FIGS. 10A-E show perspective illustrations of an indicator device and a system including the same, according to some embodiments. The device illustrated in FIGS. 10A-D show unidirectional food sample passage initiated by pressure, whereby the passage is opened by pressing the device, to thereby allow the food sample to enter the indicator. Once pressure is released, the passage is sealed/closed. FIGS. 10A-B shows perspective view of an indicator device without (FIG. 10A) and with (FIG. 10B) an external cover. FIG. 10C shows a perspective illustration of a system including a food package associated with the indicator device FIGS. 10D-E show a zoom-in view of a cross section of a device associated with a food package with closed passage (FIG. 10D) and open passage (FIG. 10E).

FIGS. 11A-B show perspective illustrations of an indicator device and a system including the same, according to some embodiments. The device and system illustrated in FIGS. 11A-B demonstrate unidirectional food sample passage initiated by pressure, whereby the passage is opened by pressing the device, to thereby allow a food sample to enter the indicator. Once pressure is released, the passage is sealed/closed. FIG. 11A shows a perspective view of a system including an indicator device and food package and FIG. 11B shows a zoom-in view of cross section of a device associated with a food package in closed and opened passages.

FIG. 12 shows perspective illustration of an indicator device and a system including the same, according to some embodiments. The device and system illustrated in FIG. 12 show unidirectional food sample passage initiated by pressure, whereby the passage is opened by pressing the device, to open an internal opening within the food package, to thereby allow a food sample to enter the indicator. Once pressure is released, the passage is sealed/closed.

FIGS. 13A-B show perspective illustrations of an indicator device and a system including the same, according to some embodiments. The device and system illustrated in FIGS. 13A-B show unidirectional food sample passage initiated by capillary forces, facilitated by capillary tubes that fluidly connect the food product and the indicator device. Once the indictor is filled (or saturated) with a food sample, there is no further fluid flow between the indicator device and food product. FIG. 13A shows a perspective view of a system including an indicator device associated with a food package. FIG. 13B shows cross section view of internal wall of the food package of the system, including capillary passage system, facilitating unidirectional passage of food sample from the food package to the indicator device.

FIGS. 14A-B show perspective illustrations of an indicator device and a system including the same, according to some embodiments. The device and system illustrated in FIGS. 14A-B show unidirectional food sample passage facilitated by valve unit, whereby one valve in open and one is closed and by circling/turning around an activating button, the valve that was open is closed and the valve that was closed is opened. Such mechanism allows graduate pass of a food sample from food container to the indicator device in one direction (unidirectional). Once food sample has passed, the indicator device can be locked for additional passes, as required. FIG. 14A shows a cross section internal view of food package of the system, associated with the indicator device. FIG. 14B shows the passage valve unit in different configurations (opened/closed).

FIGS. 15A-C show perspective illustrations of an indicator device and a system including the same, according to some embodiments. The device and system illustrated in FIGS. 15A-C show unidirectional food sample passage from a solid/semi solid food product to an indicator device. Shown in FIG. 15A is an exemplary indicator device, configured to be placed/positioned on a food package. Shown in FIGS. 15B-C is indicator device located on solid food product. The indicator device includes a region/chamber that the food or sample from the food is penetrating through dedicated tubes/channels/capillaries;

FIGS. 16A-B—show pictograms of fresh (FIG. 16A) or autoclaved (FIG. 16B) chicken breast food samples associated with an indicator device. Discoloration of the indicator device at the indicated colony forming units (CFU) count are shown;

FIGS. 17A-D—show pictograms and corresponding microbial count of various regions of fresh chicken breast slices stored at 4° C. for 5 days. FIG. 17A—pictogram of chicken breast slices, indicating the front, vertex or rear regions; FIG. 17B—bar graphs showing the microbial count (log CFU/gr) of the various breast chicken regions; FIG. 17C—pictogram of chicken breast slices, indicating the inner (internal) or upper (external) regions; FIG. 17D—bar graphs showing the microbial count (log CFU/gr) of the breast chicken regions;

FIGS. 18A-D show pictograms of chicken breast samples packed in various types of packaging and associated with indicator devices, before and after incubation (4° C. for 2 days). Further indicated are the tested CFU (log/gr) when discoloration of the indicator is observed and the coefficient of variation (CV) percentage. FIG. 18A—chicken breast packaged with polyethylene wrap and associated with an indicator device including indicator composition of 10 ppm indigo carmine and a mediator layer; FIG. 18B—chicken breast packaged in vacuum pack and associated with an indicator device including indicator composition of 10 ppm indigo carmine and a mediator layer; FIG. 18C—chicken breast packaged in vacuum pack and associated with an indicator device including indicator composition of 10 ppm indigo carmine and a mediator layer; FIG. 18D—chicken breast packaged in vacuum pack and associated with an indicator device including indicator composition of 10 ppm indigo carmine and 5 ppm tartrazine, and a mediator layer. Shown in FIGS. 18A-D a colored indicator (“fresh” sample) and discolored indicator (“spoiled” sample).

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout. In the figures, same reference numerals refer to same parts throughout.

In some embodiments, there is provided an indicator device configured to be attached to or associate with a food product and detect spoilage thereof, the indicator device includes
(a) a substrate; and
(b) an indicator composition associated with the substrate,
wherein the substrate is configured to at least partially be in contact with the food product, said indicator composition is configured to produce a detectable signal upon reacting with a portion of the food product, or with components secreted therefrom, whereby the detectable signal is indicative of food spoilage.

In some embodiments, the substrate is non-permeable. In some embodiments, the substrate is permeable. In some embodiments, the substrate is semipermeable. In some embodiments, one face of the substrate is permeable and the opposing face is not permeable. In some embodiments, the substrate is at least partially permeable. In some embodiments, the substrate is permeable. In some embodiments, the substrate is woven. In some embodiments, the substrate is non-woven. In some embodiments, the substrate is rigid. In some embodiments, the substrate is semi-rigid. In some embodiments, the substrate is soft. In some embodiments, the substrate may be or may be made of any type of fibrous material, allowing fluid passage or absorption. In some embodiments, the substrate may include textile fibers. In some embodiments, the substrate may include naturally produced textile fibers. In some embodiments, the fibers may include cellulose. In some embodiments, the substrate may include cotton. In some embodiments, the substrate may be made of cotton. In some embodiments, the substrate may be made of plastic, polypropylene, polyethylene, combination of plastic and fibers or any other suitable material. In some embodiments, the substrate may be made of polypropylene non-woven fabrics. In some embodiments, the substrate is woven. In some embodiments, the substrate may be made or include: textile fibers, plastic, nylon, woven fibers, woven fabrics, non-woven fabrics, artificial polymers, biodegradable polymers, cotton, wool, linen, oligo-sugars, polysaccharides, gels, modified starch, metals, aluminum, paper, carton, wood, plant material, clay, silk, adhesives, ointment, vax, filter paper, oil, leather, cellulose/starch chitosan/PVA (optionally associated with a fabric), cellulose/PVA cross-linked film (optionally associated with a fabric), carbon and graphite felts, carbon fibers, acetic acid, glycerin, or any combination thereof. Each possibility is a separate embodiment. In some embodiments, the substrate may include a combination of a materials, such as, for example, cotton treated/coated with starch (such as, starch chitosan), film coated cotton, acetic acid coating, glycerin coating, PVA coating, and the like, or any combination thereof.

In some embodiments, the substrate may be further coated with an additional layer (for example, a film layer), that may be made, for example, from PVA (Polyvinyl alcohol), acetic acid, glycerin, lecithin, chitosan, and the like, or any combination thereof. In some embodiments, such coating layer may at least partially prevent or diminish indictor leakage from the substrate to the food product.

In some embodiments, the substrate may include one or more additional layers that may be made of, for example, but not limited to: plastic, polypropylene, polyethylene, combination of plastic and fibers, polypropylene non-woven fabrics, woven material, textile fibers, plastic, nylon, woven fibers, woven fabrics, non-woven fabrics, artificial polymers, biodegradable polymers, cotton, wool, linen, oligo-sugars, polysaccharides, gels, modified starch, metals, aluminum, paper, carton, wood, plant material, clay, silk, adhesives, ointment, vax, filter paper, oil, leather, cellulose/starch chitosan/PVA (optionally associated with a fabric), cellulose/PVA cross-linked film (optionally associated with a fabric), carbon and graphite felts, carbon fibers, or any combination thereof. Each possibility is a separate embodiment. In some embodiments, the one or more additional layers may further include or be treated with a cationic agent and/or NaOH. In some embodiments, the additional layer is a mediator layer. In some embodiments, the one or more additional layers are configured to collect/absorb/retain residual indicator leaking from the indicator composition associated with the substrate towards the food product, in cases in which such residual leakage occurs. In some embodiments, at least one of the additional layers is positioned between the food product/food packaging and the substrate. In some embodiments, the one or more additional layers may be transiently, reversibly, or permanently associated with the substrate. In some embodiments, an additional layer may include a top cover/patch, configured to facilitate placement/association of the device and the food product/food packaging. In some embodiments, the association between the substrate and the one or more additional layers may be facilitated by any means known in the art, including, for example, stitching, adhering, gluing, welding, and the like. Each possibility is a separate embodiment.

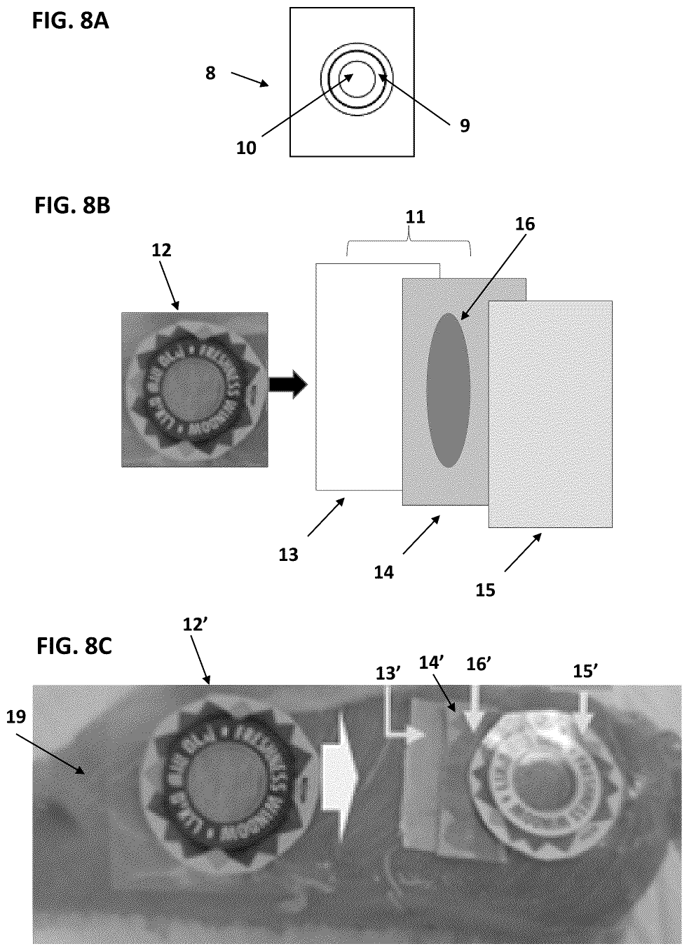

Reference is made to FIGS. 8A-B, which show exemplary illustrations of indicator device, according to some embodiments. Shown in FIG. 8A is exemplary indicator device 8, which may be essentially flat, for example, in the form of a sticker or patch. Indicator device 8 includes a substrate 9 which includes/is associated with/attached to/linked to indicator composition 10. Reference is now made to FIG. 8B, which shows a pictogram of exemplary indicator device 12 and illustration of various parts/portions/layers thereof, including: substrate 14 which is associated with indicator composition 16, optional additional layer 13 (also referred to as mediator layer), which is associated with the substrate (for example, by gluing, adhering, stitching, welding). Additional layer 13 is configured to at least partially prevent any leakage of indicator composition to the food product. In some embodiments, additional layer 13, is positioned between the food product/food packaging and the substrate. Further shown is top layer/cover 15, which is configured to facilitate placement/association of the device and the food product/food packaging, for example, by sticking/adhering thereto. Reference is now made to FIG. 8C, which shows a pictogram of indicator device 12′, placed on a food product (chicken breast 19). Right hand panel shows separate layers/part of indictor device 12′, including: mediator layer 13′, substrate 14′, which is associated with indicator composition 16′, and top layer/cover 15′, shown in the form of a sticker, configured to adhere/place/fix the device to the food product.

As used herein, “Cotton” usually refers to a natural polymer derived from cotton plants and is primarily made of cellulose. Cotton has the ability to absorb liquids.

In some exemplary embodiments, the indicator composition may include at least one indicator and at least one additional agent, such as, but not limited to: Sera Fast GMX, Denitex BC, glycidyltrimethylammonium chloride, betaine, casein, choline chloride, lecithin, chitosan, dicyandiamide, melamine, urea, biotin, casein, betaine chloride, proteins, such as albumin and casein, and the like, or any combinations thereof. Each possibility is a separate embodiment. In some embodiments, the agent is a cationic agent. In some embodiments, some of the agents may be used as adjuvants for facilitating binding of other agents to the substrate. For example, in some embodiments, materials, such as, dicyandiamide, melamine, citric acid, phosphoric acid, sodium carbonate, sodium bicarbonate, sodium hypophosphite, Polyvinyl Pyrrolidone, Aluminosilicate clay, urea, and the like, may be used as adjuvants, pH adjusters and/or catalyst, during preparation/fabrication of the device. In some embodiments, the indicator composition does not include quaternary salt, such as 2,3-epoxypropyltrimethylammonuim chloride. In some embodiments, such salts are used for pretreatment of cotton or cellulose and substrate, any free residues of quaternary salts are thoroughly washed before linking reaction with the indicator. In some embodiments, the indicator composition may have a pH above 7.

According to some embodiments, the production/preparation/fabrication of the device may include one or more steps, including, for example, pre-treating of the substrate (for example, by cationic agents), coating the substrate (with one or more suitable coating solutions), associating/linking/binding the indictor composition to the substrate, associating one or more additional layers, drying, sterilizing (for example, by autoclave); and the like, or any combination of steps thereof. The type, order, length of such method steps are determined based on the type of device, type of substrate, type of indictors in the composition, and the like.

According to some embodiments, sterilization of the indicator device may include any suitable sterilization methods, including, for example, cold pasteurization (that uses an electron beam accelerator, E-Beam), High Pressure Processing (HPP), Soaking in ethanol 70%, Treatment with ethanol or ethanol-water steam, UV treatment, IR treatment, Ozonation (for example, by soaking in ozonated water) High temperature sterilization (for example, at 300° C. for very short time period (minutes); Ultrasound treatment, and the like, or any combination thereof. Each possibility is a separate embodiment.

In some embodiments, the substrate may include cellulose and the indicator composition may include at least one indicator and at least one cationic agent. In some embodiments, the cationic agent comprises quaternary ammonium compounds. In some embodiments, the cationic agent may include 2,3-epoxypropyltrimethylammonuim chloride. In some embodiments, said permeable substrate comprises cellulose and the indicator composition may have a pH above 7. In some embodiments, the substrate is pretreated with a cationic agent, such as, 2,3-epoxypropyltrimethylammonuim chloride and NaOH, and then dyed/associate with an indictor composition (for example, indigo carmine).

In some embodiments, the substrate may be further coated with an additional layer (for example, a film layer), that may be made, for example, from PVA, acetic acid, glycerin, lecithin, chitosan, and the like, or any combinations thereof. Each possibility is a separate embodiment. Additionally or alternatively, in some embodiments, the substrate may be attached/associated with an additional layer, that may include, for example, cotton fiber pretreated with cationic agent and NaOH.

In some embodiments, the substrate may further include or be coated with amino acids, proteins and/or lipids. In some embodiments, the permeable substrate may include wool, cotton, silk, paper and/or mixed fabrics. In some embodiments, said permeable substrate may be made of wool, cotton, silk, cellulose (for example, in the form of a filter paper) and/or mixed fabrics. In some embodiments, the substrate may be coated with a coting mixture. In some exemplary embodiments, a cotton fabric substrate may be coated with a coating mixture which includes PVA and chitosan. In some exemplary embodiments, a cellulose coffee filter substrate may be coated with Betaine Solution (for example, 8%).

As used herein, “Wool” is a textile fiber obtained from animals, primarily from sheep. Wool primarily includes protein, such as, keratin, and may further includes lipids, which makes is different from other natural fibers, such as, cotton. Wool has the ability to absorb liquids.

In some embodiments, the substrate may include synthetic textile fibers, such as, for example, plastic fibers, nylon fibers, polypropylene fibers, Polypropylene non-woven fabric, and the like. In some embodiments, synthetic fibers may include a combination of synthetic textile fibers and natural textile fibers, such as, cotton, wool, silk, and the like, at any suitable/desired ranges. In some embodiments, synthetic fibers may include a combination of Polyurethane/cotton fibers, for example, in the ranges of Polyurethane/cotton 10%/90% to 90%-10%. In some embodiments, synthetic fibers may include a combination of any type of suitable materials.

According to some embodiments, the indicator composition is associated with the permeable substrate, by being attached or linked thereto. In some embodiments, the attachment of the indicator composition and the permeable substrate is such that there is minimal/minor leakage of indicator composition from the substrate (for example, less that about 10000 ppm, less than about 1000 ppm, less than about 100 ppm. less than about 10 ppm, less than about 1.0 ppm, less than about 10%, less than about 5%, less than about 1%, and the like). In some embodiments, the attachment of the indicator composition and the permeable substrate is such that there is essentially no detectable or no leakage of indicator composition from the substrate, as further exemplified hereinbelow. In some embodiments, the indicator composition is irreversibly associated with the permeable substrate. In some embodiments, the indicator composition is covalently attached to the permeable substrate. In some embodiments, the indicator is irreversibly attached to the permeable substrate. In some embodiments, the indicator is reversibly attached to the substrate.

In some embodiments, at least one indicator composition is associated with the substrate. In some embodiments, more than one indicator composition may associate with the substrate. In some embodiments, when a plurality (for example, at least two) indicator compositions are associated with the substrate, each indicator composition may be similar, identical or different with respect of distribution, type of indicator, composition and/or concentration. In some embodiments, when a plurality of indictor compositions are associated with the substrate, each indicator composition may be associated with discrete spatial regions of the substrate. In some embodiments, the plurality of indicator compositions may be substantially evenly or uniformly distributed on the substrate. In some exemplary embodiments, when a plurality of indicator compositions (for example, 2 indicator compositions) are used, the indicators in each composition may each be of different type (i.e., each capable of identifying or recognizing a different pathogen) and being at same or different concentrations. In some exemplary embodiments, when a plurality of indicator compositions are present (for example, 2 indicator compositions), the indicators at each composition may be of the same type, but at different concentrations, thereby enabling a quantitative (or at least semi quantitative) assessment or indication as to level of spoilage. In some embodiments, as detailed below, an indicator composition may include a plurality of indicators.

In some embodiments, the indicator composition include at least one indicator and at least one carrier. In some embodiments, the carrier may be any one or more of a solvent, an acid, a base and a diluent. In some embodiments, the carrier is oil. In some embodiments, the carrier is or includes water or aqueous solution. In some embodiments, the indicator composition may include a plurality of indicators, that may be identical, similar or different with respect of type and/or concentration. In some exemplary embodiments, when a plurality of indicators are present in the indicator composition (for example, 2 indicators), the indicators may each be of different type (i.e., each capable of identifying or recognizing a different pathogen) and being at same or different concentrations. In some exemplary embodiments, when a plurality of indicators are present in the indicator composition (for example, 2 indicators), the indicators may be of the same type, but at different concentrations In some embodiments, the indicator composition may be an emulsion of indicator and vegetable oil with one or more optional adjuvants (for example, with indicators which are not soluble in water).

As used herein, the term “about” may be used to specify a value of a quantity or parameter (e.g., pore size, molecular weight) to within a continuous range of values in the neighborhood of (and including) a given (stated) value. According to some embodiments, “about” may specify the value of a parameter to be between 80% and 120% of the given value. For example, the statement “about 1” is equivalent to the statement “between 0.8 and 1.2”. According to some embodiments, “about” may specify the value of a parameter to be between 90% and 110% of the given value. According to some embodiments, “about” may specify the value of a parameter to be between 95% and 105% of the given value.

In some embodiments, the food product is in a liquid form, e.g. milk and non-dairy/vegan milk. In some embodiments, the food product is in a semi-liquid form, e.g. soft cheese and gelatin. In some embodiments, the food product is in a solid form, e.g. chicken breast, beef, fish, (such as fish fillet), plant based food, and the like.

According to some embodiments, during the use of the device, the indicator component within the indicator composition, or the indicator composition, may at least partially or transiently (timely) contact the food product to which the indicator device is attached/associated with.

In some embodiments, the indicator composition may include non-toxic, edible, food grade indicator. In some embodiments, said indicator is selected from the group consisting of pH indicators, sulfhydryl compounds with sulphonate functional groups, and nitrogen. In some embodiments, the indicator comprises Ninhydrin, phthalocyanines, coumarins and/or trifluoro acetyl derivatives. In some embodiments, said indicator comprises azo dye. In some embodiments, said indicator comprises indigo carmine.

In some embodiments, at least one indicator is selected from microbial indicators, redox indicators and pH indicators. In some embodiments, at least one indicator is a pH indicator. In some embodiments, at least one indicator is a microbial indicator.

In some embodiments, the microbial indicator is devoid of quaternary ammonium salts and quaternary ammonium moieties.

In some embodiments, the at least one microbial indicator provides a detectable indication in the presence of a microbial population above a pre-determined threshold. In some embodiments, the at least one microbial indicator provides a detectable indication in the presence of a population of specific types/species of microorganisms/pathogens.

In some embodiments, at least one microbial indicator is selected from methyl red, methyl orange, indigo carmine, bromophenol blue, carmoisine red, tartrazine, bromocresol green, brown HT, methylene blue, Alizarin, Bromocresol Purple, Chlorophenol Red, Nitrazine Yellow, Bromoxylenol Blue, Neutral Red, methyl orange, bromothymol blue, cresol red, phenol red, methyl red, bromocresol blue, indigo carmine, carmoisine red, tartrazine, bromocresol green, methyl orange, methyl blue, sunset yellow FCF, citrus red 2, methylene blue, Brown HT, amaranth, Ponceau 4R, quinoline yellow, carmoisine, patent blue V, green S, brilliant blue, indigotine, fast green FCF, erythosine, allura red AC, orange B, scarlet GN, sudan II, acid orange 20, annatto, caramel coloring, carmine, elderberry, lycopene, paprika, turmeric, tetrazolium salts, resazurin, Ninhydrin, phthalocyanines, coumarins, trifluoro acetyl derivatives or any combinations thereof. Each possibility is a separate embodiment. In some embodiments, at least one microbial indicator comprises an azo dye.

In some embodiments, the at least one indicator is selected from bromothymol blue, cresol red, phenol red, methyl red, bromocresol blue, indigo carmine, carmoisine red, tartrazine, bromocresol green, bromophenol blue and methyl orange.

In some embodiments, the composition further comprises at least one transition metal moiety. In some embodiments, the transition metal moiety comprises Cr(III).

The term “indicator” as used herein refers to any substance capable of changing at least one property (such as, color change, spectroscopic change, colorimetric change, fluorimetry change and/or electrochemical change), with a change in a property in its environment. For example, changing color includes, without limitation, losing color (discoloration), for example, when a colored indicator becomes white, colorless or substantially transparent; and gaining color, for example, when a white, colorless or substantially transparent indicator becomes colored. Environmental properties may include for example, pH, oxygen amount of microbial bacterial population, and/or type of microbial bacterial population. Accordingly, in some exemplary embodiments, pH indicators and microbial indicators are examples of materials used as indicators in the devices of the current invention. As a change in the pH of food products may be caused when a threshold amount of microbes, or microbial by-products (e.g. nitrates, nitrites, sulfur and sulfates) are produced, there may be an overlap between microbial indicators and pH indicators. Exemplary indicators are colored conjugated organic molecules, which change their color in response to a change in pH, which leads to alternation in the pi conjugation of the indicator molecule; and colored conjugated organic molecules, which change their color in response to an enzymatic or microbial reaction, which leads to alternation in the pi conjugation of the indicator molecule.

It is noted that while the microbes may or may not be harmful, the waste products of bacteria may be unpleasant to taste or may even be harmful.

In some embodiments, pH indicator provides a detectable mark within any pH applicable to the indication of freshness or spoilage of food products. In some embodiments, the pH indicator provides a detectable mark within a pH range of 3.0 to 9.0. In some embodiments, the pH indicator provides a detectable mark within a pH range of 4.0 to 9.0. In some embodiments, the pH indicator provides a detectable mark within a pH range of 5.0 to 9.0. In some embodiments, the pH indicator provides a detectable mark within a pH range of 8.0 to 9.0. In some embodiments, the pH indicator provides a detectable mark within a pH range of 7.5 to 8.0. In some embodiments, the pH indicator provides a detectable mark within a pH range of 7.0 to 7.5. In some embodiments, the pH indicator provides a detectable mark within a pH range of 6.3 to 7.0. In some embodiments, the pH indicator provides a detectable mark within a pH range of 5.7 to 6.3. In some embodiments, the pH indicator provides a detectable mark within a pH range of 5.0 to 5.7.

In some embodiments, the microbial indicator provides a detectable indication in the presence of an amount of bacteria above a certain threshold.

In some embodiments, the threshold as determined by the United States Food and Drug Administration (FDA) which promotes adherence to a food spoilage safety standard of approximately 10⁵-10⁷ (for example, 10 million) colony forming units (CFU) per gram, or CFU/g. Accordingly, food products with measured microbial levels above the FDA standard are considered unsafe for consumption and should therefore be immediately discarded. In some embodiments, any type of standard for food spoilage may be determined based on the number, concentration, amount and/or type of microorganism in a food sample.

In some embodiments, the microbial indicator provides a detectable indication in the presence of compounds associated with the presence of an amount of microbes (such as, bacteria, fungi, and the like) above a certain threshold.

In some embodiments, microbial indicators are not anti-microbial materials. In some embodiments, the compounds are the result of microbial decomposition of the food product. In some embodiments, the compounds are carboxylic acids. In some embodiments, the compounds comprise lactic acid and/or butyric acid. In some embodiments, the compounds are amines, nitrates, nitrites, sulfur and/or sulfates. In some embodiments, the compounds include products of proteins and amino-acids decomposition, such as, putrescine, cadaverine, ammonia. In some embodiments, the compounds are volatile compounds, including, for example, 1-octanol, 1-decanol, 1-dodecanol, 2-heptanone, 2-nonaone, 2-undecanone, 3-methyl-butanoic acid, 3-methylbutanal, 3-hydroxy-2-butanone, indole. In addition,

In some embodiments, the microbial population may include any type of bacterial population, including, for example, aerobic, anaerobic, psychotropic and/or mesophilic microorganisms. In some embodiments, the microbial population includes pathogenic bacteria. In some embodiments, the microbial population may include any type of microorganisms that may affect food spoilage, such as fungus (including, yeast and mold). For example, Paenibacillus is a spore-forming bacterium that is found in spoiled milk. It is responsible for spoiling milk and causes curdling as well. Paenibacillus also contributes to off flavors in a variety of other foods. Paenibacillus may withstand the extreme conditions of pasteurization and subsequent cooling in its spore state, allowing it to survive in milk and other foods.

In some embodiments, the microbe population comprises fungi. In some embodiments, the fungi comprise Saccharomyces cerevisiae and/or Hansenula anomala. In some embodiments, the microbe population comprises yeast.

In some embodiments, the at least one microbial indicator is selected from the group consisting of methyl red, methyl orange, bromophenol blue, indigo carmine, carmoisine red, tartrazine, bromocresol green and combinations thereof.

In some embodiments, the composition of the at least one indicator includes an active ingredient that is adapted to change color within a defined color range that is dependent upon the concentration of compounds associated with food spoilage.

In some embodiments, the at least one indicator is a colorimetric indicator.

The term “colorimetric indicator” as used herein refers to an indicator capable of changing color, including, for example, the change from colored to colorless (e.g. white), in response to sensing, e.g. interacting with, compounds characteristic of spoiled food, or sensing conditions associated with such spoilage (for example, change in pH, O₂ levels, etc.).

In some embodiments, the colorimetric indicator may provide a color indication which includes any one or more of a first color indicating freshness (e.g. green), a second color indicating spoilage (e.g. red) and a third color indicating that the food product is about to be spoiled (e.g. yellow). The latter may provide a specific date, or time window, during which the food product is still fresh but by after the specified date, or by the end of the time window, the food product will be considered spoiled.

In some embodiments, the detectable indication may include any one or more of the following indications: an indication of freshness, an indication of spoilage and a warning indication. The latter may be an alert, some time prior to spoilage, e.g. a few days before the food product is spoiled, thereby providing the user an indication of the time remains until the food product losses its freshness and/or becomes spoiled.

In some embodiments, the detectable indication is a colored detectable indication. In some embodiments, the detectable indication comprises a change in color within the visual spectrum.

Table 1 provides an exemplary pH transition range in aqueous environment of several indicators.

TABLE 1

| | aqueous pH | | |
|---|---|---|---|
| | transition | | |
| Indicator | range | color change | CAS |
| Cresol Red | 7.2-8.8 | yellow to reddish | 1733-12-6 |
| | | purple | |
| Alizarin | 5.5-6.8 | yellow to violet | 72-48-0 |
| Bromocresol Purple | 5.2-6.8 | yellow to purple | 115-40-2 |
| Chlorophenol Red | 5.2-8.8 | yellow to red | 4430-20-0 |
| Nitrazine Yellow | 6.0-7.2 | yellow to bright blue | 5423-07-4 |
| Bromothymol Blue | 6.0-7.6 | yellow to blue | 34722-90-2 |
| Bromoxylenol Blue | 6.0-7.6 | yellow to blue | 40070-59-5 |
| Neutral Red | 6.8-8.0 | red to yellow | 553-24--9 |
| Phenol Red | 6.8-8.2 | yellow to red | 34487-61-1 |

In some embodiments, at least one indicator is selected from the group consisting of methyl red, methyl orange, bromophenol blue, indigo carmine, carmoisine red, tartrazine and bromocresol green. Each possibility is a separate embodiment of the invention.

In some embodiments, the indicator device is biocompatible. In some embodiments, the indicator device is non-antimicrobial (microbe compatible). In some embodiments, the at least one indicator in the indicator composition of the device is biocompatible. In some embodiments, the at least one indicator is non-antimicrobial (microbe compatible). In some embodiments, the at least one indicator is devoid of quaternary amines and quaternary amine moieties. In some embodiments, the at least one indicator is devoid of quaternary ammonium salts and quaternary ammonium moieties. According to some embodiments, an indicator or mixture of indicators do not include quaternary amine salts as part of composition so they do not cat as antibacterial agent and inhibit/delay the spoilage. In some embodiments, specific epoxy quaternary salt (such as, 2,3-102Epoxypropyltrimethylammonium chloride, betaine, lecithin) are utilized to link indicator (such as a dye) to the substrate (such as, cotton/cellulose/wool/nylon/silk/paper, and the like) backbone.

In some embodiments, certain indicators may be specific to certain pathogens.

In some embodiments, there is provided a food product comprising an indicator device attached thereto, wherein the indicator device comprises
(a) a substrate; and
(b) an indicator composition, at least a portion thereof is associated with the substrate,
wherein said substrate is configured to be in contact (directly or indirectly, transiently or permanently) with the food product (or at least a food sample), said indicator composition is configured to produce a detectable signal upon reacting with a portion of the food product, or with components secreted therefrom, wherein the signal is indicative, for example, of food spoilage.

In some embodiments, there is provided a method for detecting food spoilage of a food product, the method comprising the steps of: placing an indicator device as disclosed herein in a contact (for example, fluid (gas or liquid)) with a food product, wherein the indicator device comprises a substrate; and an indicator composition attached to the permeable substrate; allowing at least a portion of the food product, or components secreted therefrom to contact said indicator composition, and to react therewith; and determining that said food product is spoiled upon detecting the detectable signal from the indicator device.

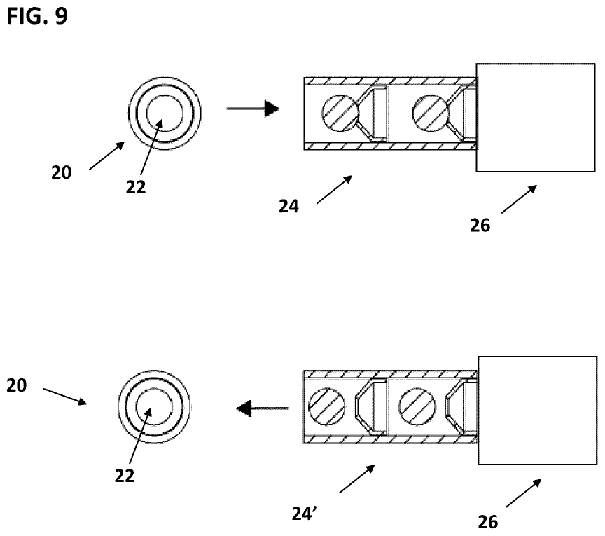

Reference is now made to FIG. 9 which shows perspective illustrations of an indicator device, allowing unidirectional food sample passage, according to some embodiments. Shown in FIG. 9 is an exemplary indicator device 20, which is further associated with/includes passage 24, which is configured to enable a unidirectional passage of a food sample 26 to indicator 20 of the indicator device. Passage 24 can acquire at least two positions: a closed position (represented as 24), which does not allow passage from the indicator towards the food product, and an open position (represented as 24′), which allows passage from food sample 26 towards indicator 22 (in particular to indicator). The unidirectional movement is facilitated by passage 24, which, as illustrated in FIG. 9, includes an internal bearing capable of allowing unidirectional fluid passage (i.e., from food sample side towards the indicator side). The passage may be associated with, attached to, formed with, or reversibly associated with the indicator device.

In some embodiments, there is provided an indicator device configured to attach to a food package containing a food product and detect spoilage thereof, the indicator device includes
(a) a substrate including an indicator composition; and
(b) at least one passage configured to enable a unidirectional passage of a portion of the food product, or of components secreted therefrom, into the indicator device,
wherein said indicator composition is configured to produce a detectable signal upon reacting with said portion of the food product, or with said components secreted therefrom.

In some embodiments, the indicator device is impermeable. An impermeable indicator device refers to a device that does not enable transfer of components into the device, unless the at least one passage is open. In some embodiments, the device does not essentially enable transfer of components, or detectable levels thereof, from the device to the food product. In some embodiments, the indicator device is permeable.

In some embodiments, the passage is a single passage (i.e., occurring only once). In some embodiments, the passage may be repeated for any number of times. In some embodiments, the passage is unidirectional.

In some embodiments, as detailed above, the indicator composition may be irreversibly or at least over 80% thereof attached to/associated with the substrate.

In some embodiments, the at least one passage enables a unidirectional and/or a single (one time) and/or a plurality of passages of at least a portion of the food product, and/or of components secreted therefrom, to/into/towards the indicator device

In some embodiments, the at least one passage comprises at least one capillary.

In some embodiments, the at least one passage comprises a plurality of capillaries.

The term “plurality” as used herein, refers to more than one.

Using one or more capillaries enables spontaneous one-way flow of a liquid from the food product (i.e., “food sample”) into the indicator device driven by capillary forces. The liquid may be the food product (or a portion thereof) when the food product is a liquid, such as, milk, or may be liquids secreted from the food product, such as, for example, meat (beef, poultry, fish, ham, and the like). The food sample conveyed to the indicator would contain the microbial signature of the food product, thereby enabling identification/determination/indication thereof, when interacting with the indicator. In some embodiments, the food sample may contain at least one reactant molecule or entity reflecting food spoilage. In some embodiments, the food sample may contain one or more reactant molecules produced in the food product, indicative of food spoilage. In some embodiments, the food sample contains a representative microbial signature of the food product.

In some embodiments, the indicator composition is attached to the substrate and the at least one passage is a closed passage which can be transiently opened when required.

The term ‘transiently opened’ as used herein refers to a brief, one-time opening, that enables transition of a food portion or of components secreted therefrom (food sample), into the indicator device and thereafter seals again the at least one passage, thereby preventing further transition into the indicator device.

The terms ‘brief’ and ‘briefly’ as used herein refer to a short time period during which a passage between the food product and the indicator device, is enabled. Stated otherwise, these terms refer to a short time period during which the passage between the food product and the indicator device, is open. The short time period may be in the range of millisecond, seconds, minutes to one second. For example, the short time may be in the range of 1 second-2 minutes.

In some embodiments, the closed passage which can be transiently opened when required includes a single use safety syringe component, configured to withdraw a food portion or components secreted from the food product, into the indicator device, once. In some embodiments, the close passage comprises a single use safety syringe component, configured for a single withdrawal of a food portion or components secreted from the food product, into the indicator device. In some embodiments, the safety syringe component includes, but is not limited to, through-push retract breakable safety syringe, rotary plunger retractable safety syringe and release-ring breakable safety syringe.

In some embodiments, the close passage which can be transiently opened when required includes a displacing element, configured to briefly open the close passage through displacement, thereby enable transition of a food portion or components secreted from the food product, into the indicator device.

In some embodiments, the displacement is rotation.

In some embodiments, the indicator composition is encapsulated within the substrate. In some embodiments, the substrate is an impermeable capsule. In some embodiments, the passage is a close passage configured to be transiently opened when required. In some embodiments, wherein the close passage comprises an infiltrating unit, configured to briefly open the close passage and infiltrate the capsule, thereby enable transition of a food portion or components secreted from the food product, into the indicator device.

In some embodiments, there is provided a food packaging including a food product, the packaging includes an indicator device attached thereto, wherein the indicator device includes a substrate having an indicator composition.

In some embodiments, there is provided a food packaging configured to hold/include/wrap a food product, the packaging includes an indicator device attached thereto, wherein the indicator device includes
(a) a substrate having an indicator composition; and
(b) at least one passage configured to enable a unidirectional passage of a food sample (portion of the food product, or of components secreted therefrom), into the indicator device,
wherein the indicator composition is configured to produce a detectable signal upon reacting with said food sample, wherein the detectable signal is indicative of the freshness of the food product.

The detectable signal may indicate that the food is spoiled or not spoiled, for example, due to presence/amount of any type of microorganism, such as, bacteria, fungi, yeast, mold, pathogenic or non-pathogenic, depending on the type of activity of the indicator composition with the food product. Thus, the food product or the indicator device may further include an index, such as, a numerical or color scale, showing the correlation between the detected signal and freshness and/or spoilage.

Thus, in some embodiments, the detectable signal indicates that the food product is spoiled. In alternative embodiments, the detectable signal indicates that the food product is not-spoiled. In some embodiments, the detectable signal indicates that the food is fresh. In some embodiments, the detectable signal is qualitative. In some embodiments, the detectable signal is quantitative. In some embodiments, the detectable signal is qualitative and/or quantitative. In some embodiments, the detectable signal is indicative of the type and/or species and/or of the microbial population. In some embodiments, the detectable signal may be indicative if the microbe is a pathogen.

In some embodiments, there is provided a method for detecting food spoilage or freshness of a food product, the method includes the steps of placing/positioning an indicator device in contact with a food product, wherein the indicator device comprises a substrate comprising at least one indicator composition; and at least one passage configured to enable a single/unidirectional passage of a food sample (portion of the food product, or of components secreted therefrom), into the indicator device; allowing the food sample to contact said indicator composition within the indicator device, through the at least one passage; and determining whether said food product is spoiled based on a detecting a detectable signal from the indicator device, wherein the signal is indicative of food spoilage or freshness.

In some embodiments, there is provided a method for detecting food spoilage or freshness of a food product, the method includes the steps of placing/positioning an indicator device in contact with a food product, wherein the indicator device comprises a substrate comprising an indicator composition; and at least one passage configured to enable a single or unidirectional passage of a food sample (a portion of the food product, or of components secreted therefrom, into the indicator device); allowing the food sample to contact the indicator composition within the indicator device, through the at least one passage; and determining whether said food product is spoiled upon detecting the detectable signal.

According to some embodiments, there is provided a system for detecting food spoilage of a food product, the system includes: at least one food packaging configured to hold a food product; and an indicator device associated with the food packaging.

According to some embodiments, the food product may be selected from fluid food product, liquid food product, soft food product, semi-hard food product, hard food product and aqueous food product. Each possibility is a separate embodiment.

According to some embodiments, the food packaging (also referred to as food package) may be is selected from any type of suitable packaging including, for example, but not limited to: a container, a vial, a box, a tray, a can, a palette, a bag, a wrap, and the like. In some embodiments, the food packaging may be made of any suitable material or combination of materials, including, for example, plastic, cardboard, carton, metal, glass, nylon, cotton, fabric, and the like, or any combination thereof. Each possibility is a separate embodiment. In some embodiments, the packaging is a nylon (saran) wrap. In some embodiments, packages may be at any condition, for example, they may contain oxygen or not, may be vacuumed or not, may include controlled environment of mixed portions of nitrogen and oxygen and like, or any combinations thereof.

According to some embodiments, the indicator device may be integrally formed with the food packaging. For example, the indicator device may be incorporated with/into the food packaging during or after manufacturing of the food packaging.

According to some embodiments, the indicator device may be permanently associated with the food packaging.

According to some embodiments, the indicator device may be transiently associated with the food packaging.

According to some embodiments, the indicator device may be associated with an external region of the food packaging.

In some embodiments, the indicator device may be the food packaging itself. For example, the indicator device may include a nylon substrate comprising a suitable indicator, said nylon substrate is used as a food package, by, for example, being wrapped round the food.

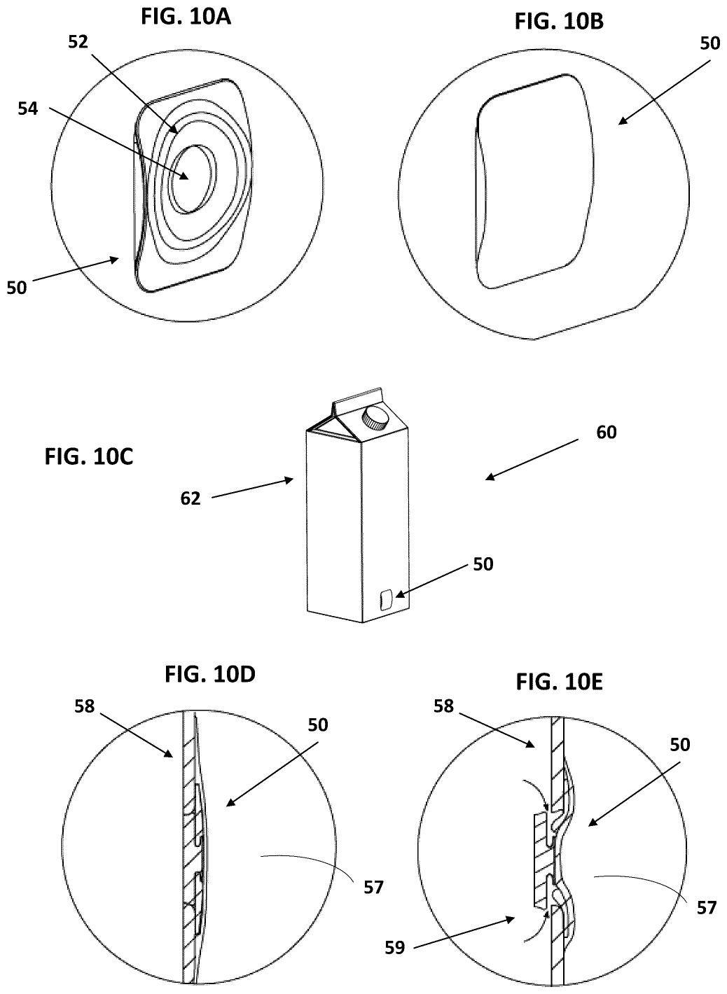

Reference is now made to FIGS. 10A-E which show perspective illustrations of an indicator device and a system including the same, according to some embodiments. The device and system illustrated in FIGS. 10A-E show unidirectional food sample passage initiated by pressure, whereby the passage is opened by pressing the device, to thereby allow the food sample to enter the indicator. Once pressure is released, the passage is sealed/closed, or vice versa. FIG. 10A shows a perspective view of exemplary indicator device 50, which includes substrate 52 and indicator composition 54. The device may further include an external cover, as shown in FIG. 10B. Shown in FIG. 10C is system 60, which includes food package 62 (shown in FIG. 10C as exemplary liquid container), and indicator device 50 associated therewith. In the example shown in FIG. 10C, the indicator device is associated with a front wall of the food package, at a lower region/portion thereof. Shown in FIGS. 10D-10E, arc zoom-in internal views of a cross section of device 50, wherein the passage portion is associated with internal wall 58 of food package 62. In FIG. 10D, the passage is closed and when the indicator device is being pressed on an outer region thereof (e.g., side 57, externally to the food package) as shown in FIG. 10E, inner region of the passage is opened, allowing passage of food sample 59 towards the indicator device 50. Thus, once the pressure is released, the passage may be closed/rescaled, thereby allowing unidirectional pass/flow of food sample from the food package towards the indicator device, and in particular, to the indicator composition, allowing interaction therewith, to provide indication regarding food freshness/spoilage.

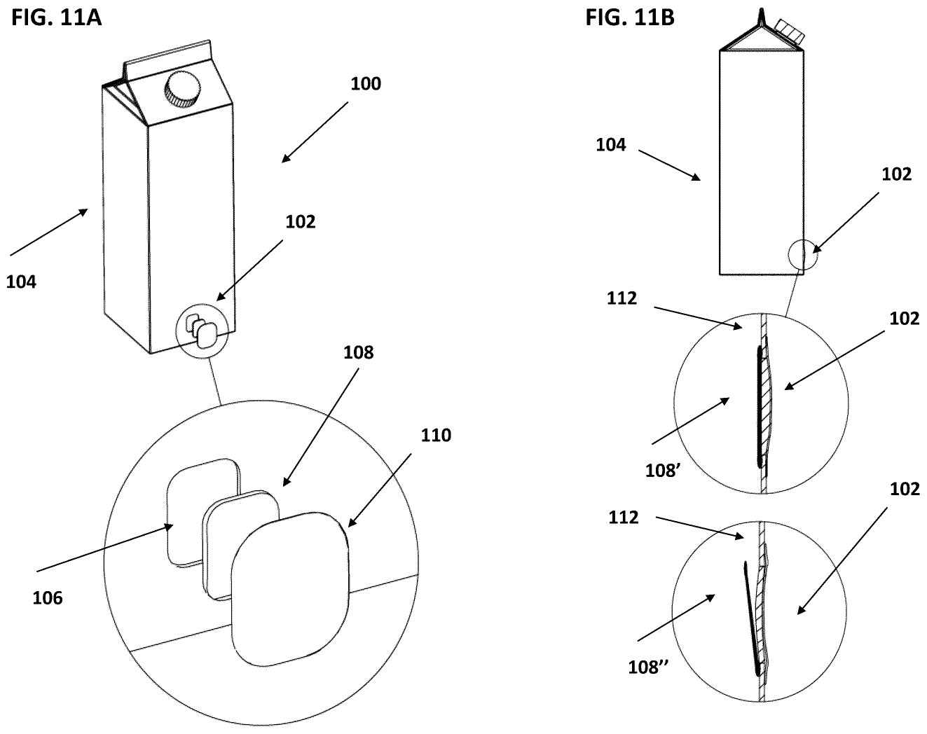

Reference is now made to FIGS. 11A-B which show perspective illustrations of an indicator device and a system including the same, according to some embodiments. The device and system illustrated in FIGS. 11A-B demonstrate unidirectional food sample passage initiated by pressure, whereby the passage is opened by pressing the device, to thereby allow a food sample to enter the indicator. Once pressure is released, the passage is sealed/closed, or vice versa. FIG. 11A shows a perspective view of system 100, including food package 104, and indicator device 102 associated therewith. In the example shown in FIGS. 11A-B, the food package is in the form of a carton and the food product may be any suitable food product, such as, liquid food product (for example, milk). In the example shown in FIGS. 11A-B, indicator device 102 is associated with a front wall of the food package, at a lower region/portion thereof. Further shown is a zoom-in view of device 102 associated with food package 104. As shown, the device includes a substrate and indicator composition 110 and passage 108 (allowing unidirectional food passage, as detailed below), and is fitted over an opening 106 in the wall of food package 104. FIG. 11B shows a side zoom-in view of cross section of a device and food package, showing internal wall 112 of food package, whereby passage 108′ is in a closed position, scaling opening 106, and upon external pressure applied on device 102, the passage 118″ assumes an open position, allowing food passage from the internal cavity/volume of the food package to indicator device 102, and in particular to the indicator composition. Thus, once the pressure is released, the passage may be closed/resealed, thereby allowing unidirectional pass/flow of food sample from the food package towards the indicator device, and in particular, to the indicator composition, allowing interaction therewith, to provide indication regarding food freshness/spoilage.

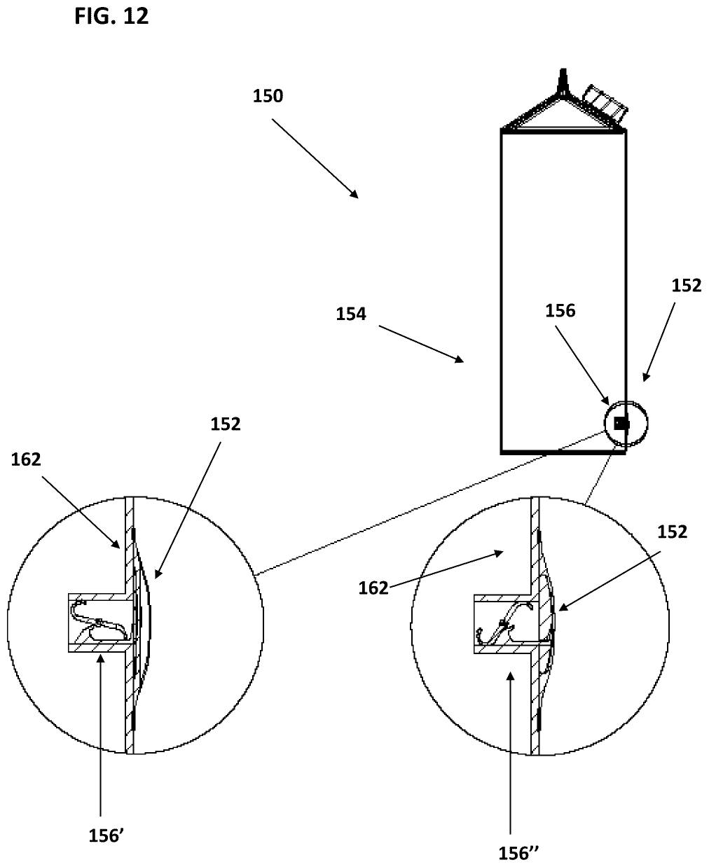

Reference is now made to FIG. 12, which shows perspective illustration of an indicator device and a system including the same, according to some embodiments. The device and system illustrated in FIG. 12 show unidirectional food sample passage initiated by pressure, whereby the passage is opened by pressing the device, to open an internal opening within the food package, to thereby allow a food sample to enter the indicator. Once pressure is released, the passage is sealed/closed, or vice versa. FIG. 12 shows a perspective view of system 150, including food package 154, and indicator device 152 associated therewith. In the example shown in FIG. 12, the food package is in the form of a carton and the food product may be any suitable food product, such as, liquid food product (for example, milk). In the example shown in FIG. 12, indicator device 102 is associated with a front wall of the food package, at a lower region/portion thereof. Further shown are side zoom-in views of cross section of a device and food package, showing internal wall 162 of food package 154, whereby passage 156′ of indicator device protrude into the internal volume/cavity of food package 154, and can assume a closed (sealing) position 156′ or an open position 156″. External pressure applied on device 152, facilitates the position change of passage 156, whereby the open position, allows food passage from the internal cavity/volume of the food package to indicator device 152, and in particular to the indicator composition thereof. Thus, once the pressure is released, the passage may be closed/rescaled, thereby allowing unidirectional pass/flow of food sample from the food package towards the indicator device, and in particular, to the indicator composition, allowing interaction therewith, to provide indication regarding food freshness/spoilage.

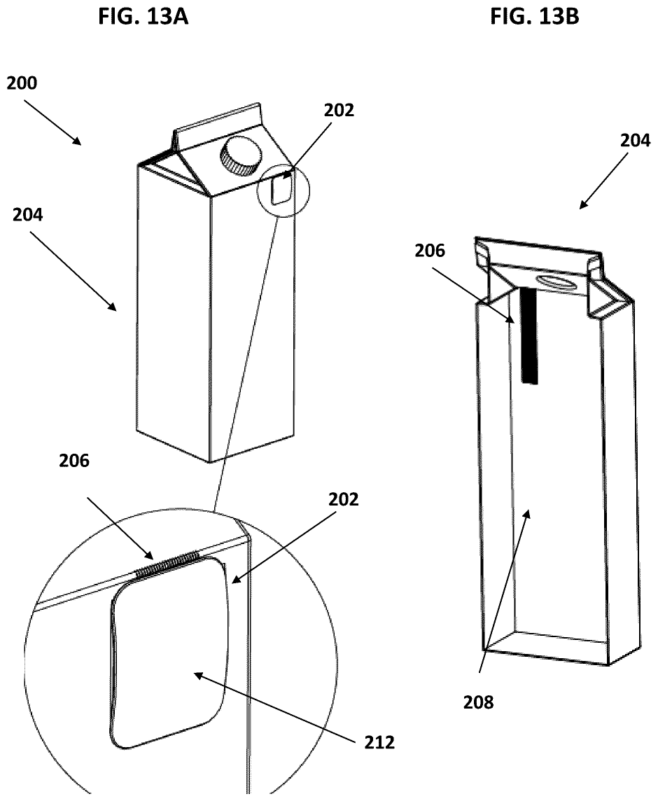

Reference is now made to FIGS. 13A-B, which show perspective illustrations of an indicator device and a system including the same, according to some embodiments. The device and system illustrated in FIGS. 13A-B show unidirectional food sample passage initiated by capillary forces, facilitated by capillary tubes that fluidly connect the food product and the indicator device. Once the indictor is filled (or saturated) with a food sample, there is minimal or no further fluid flow between the indicator device and food product. FIG. 13A shows a perspective view of system 200, including an indicator device 202 associated with food package 204. In the example shown in FIG. 13A, the food package is in the form of a carton and the food product may be any suitable food product, such as, liquid food product (for example, milk). In the example shown in FIG. 13A, indicator device 202 is associated with a front wall of the food package, at an upper region/portion thereof. Further shown in FIG. 13A is a zoon-in view of device 202, showing substrate and indicator composition 212 and a portion of passage 206. FIG. 13B shows internal view of a cross section of food package 204, including capillary passage system 206, facilitating unidirectional passage of food sample from the internal volume 208 of food package to the indicator device, located on the external wall/surface of the food package. Thus, the capillary passage allows unidirectional pass/flow of food sample from the food package towards the indicator device, and in particular, to the indicator composition, allowing interaction therewith, to provide indication regarding food freshness/spoilage.

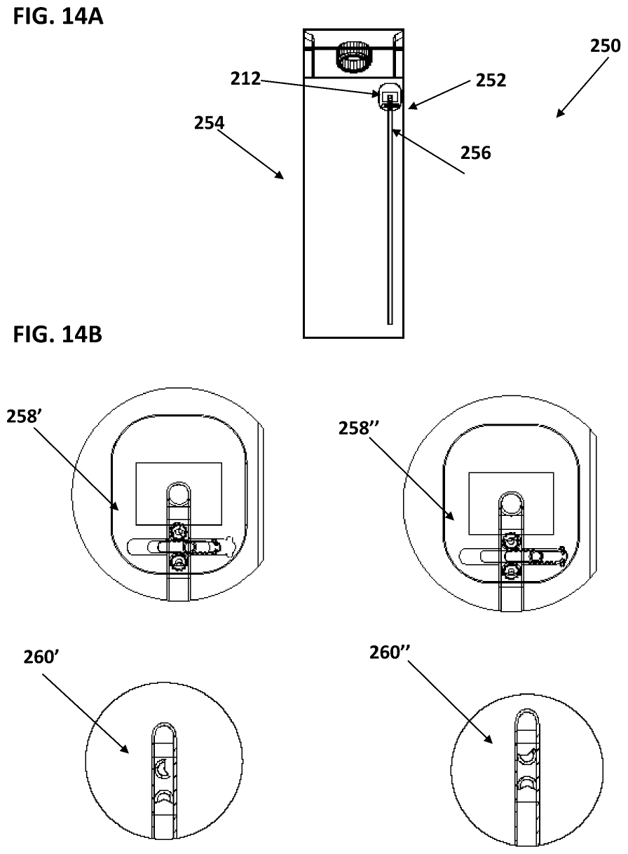

Reference is now made to FIGS. 14A-B which show perspective illustrations of an indicator device and a system including the same, according to some embodiments. The device and system illustrated in FIGS. 14A-B show unidirectional food sample passage facilitated by valve unit, whereby one valve in open and one is closed and by circling/turning around an activating button, the valve that was open is closed and the valve that was closed is opened. Such mechanism allows graduate pass of a food sample from food container to the indicator device in one direction (unidirectional). Once food sample has passed, the indicator device can be locked for additional passes, as required. FIG. 14A shows a cross section internal view of food package 254 of system 250, associated with indicator device 252. In the example shown in FIG. 14A, the food package is in the form of a carton and the food product may be any suitable food product, such as, liquid food product (for example, milk). In the example shown in FIG. 14A, indicator device 202 is associated with a front wall of the food package, at an upper region/portion thereof. Further shown in FIG. 14A, are back region of substrate and indicator composition 262 and passage 256. FIG. 14B shows the passage valve units 258 or 260 of passage 256, at different configurations, in particular, at an open configuration (258′/260′) or closed configuration (258″ or 260″). The change in configuration of the valve units is facilitated, for example, by manual turning of external button at the surface of indicator device 252. Thus, the valve unit passage allows unidirectional pass/flow of food sample from the food package towards the indicator device, and in particular, to the indicator composition, allowing interaction therewith, to provide indication regarding food freshness/spoilage.

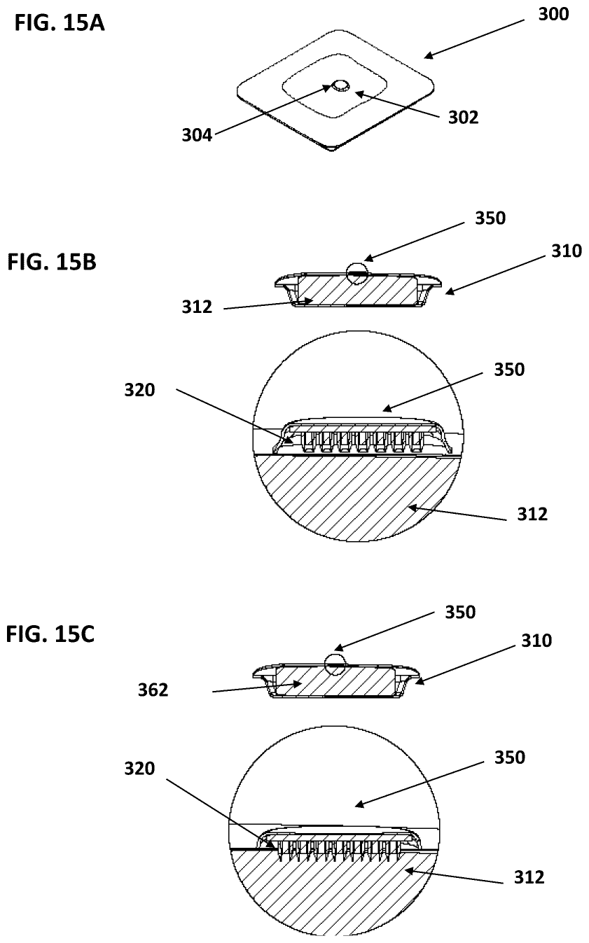

Reference is now made to FIGS. 15A-C, which show perspective illustrations of an indicator device and a system including the same, according to some embodiments. The device and system illustrated in FIGS. 15A-C show unidirectional food sample passage from a solid/semi solid food product to an indicator device. Shown in FIG. 15A is an exemplary indicator device 300, configured to be placed/positioned on a food package. Device 300 includes substrate 302 and indicator composition 304, associated/linked/attached to the substrate. Shown in FIGS. 15B-C is indicator device 350 located/placed on food package 310, holding solid food product 312 (for example, meat). Device 350 includes a substrate and indicator composition (similar to device 300) and further includes or be associated with passage 320. Passage 320 is shown is the zoom-in view, whereby passage 320, is configured to allow food sample movement from the food towards the indicator composition of the indicator device. The passage is shown in the form of tubes/channels/capillaries, wherein the interaction between the food sample and the food passage is facilitated, by, for example, applying pressure or pressing the indicator device to establish contact between the passage and the food product. FIG. 15B shows the passage prior to engaging the food product and FIG. 15C shows the passage after engaging the food product. The passage allows unidirectional movement of food sample from the food to the indicator device, and in particular to the indicator composition. In some embodiments, the indicator device includes a region/chamber to which the food sample is penetrating/passing via the dedicated tubes/channels/capillaries passage.

According to some embodiments, the indicator device may be located/placed inside the food and the color indication may be transferred/conveyed to the surface by optical (e.g. lenses), mechanical, electronical, or chemical means.

In some embodiments, the indicator device has an upper surface/face and a bottom (lower) surface/face, wherein the upper surface is configured to face the environment and the bottom surface is configured to face the food product (or food package containing the food product). In some embodiments, the properties of the faces may be different or similar (for example, with respect of permeability).

In some embodiments, the detectable signal produced by the indicator composition is visible through the upper surface of the indicator device.

In some embodiments, the indicator device is in the form of a flat card. In some embodiments, the indicator device is in the form of a sticker. In some embodiments, the indicator device is in the form of a patch.

The term ‘sticker’ as used herein refers to a flat, relatively thin, device, preferably having an upper side and a lower side, wherein the lower side is configured to face a food product and the upper side is configured to face the environment. In some embodiments, the lower side of the stickers, or parts thereof are adhesive, or include adhesive regions that maintain the sticker in contact with the food product. In some embodiments, prior to use the adhesive side or regions are irreversibly covered with a suitable film.

In some embodiments, said food spoilage is microbial spoilage.

In some embodiments, the food sample (i.e., portion of the food product, or components secreted therefrom) comprise reactant molecule(s) or entities found or produced in the food product when the food product is undergoing spoilage. In some embodiments, the reactant molecule or entity is or represents microbial content/microbial population/microbial signature in the food sample (and hence in the food product). In some embodiments, microbial population (microbial signature) includes the type, species, amount, concentration, and the like, of microbial entities in the food product. In some embodiments, the microbial signature may change over time and may be indicative of food product spoilage.

In some embodiments, the detectable signal is indicating that the food product is not spoiled. The ‘not spoiled’ as used herein may refer to fresh food product. In some embodiments, lack of detectable signal indicates that the food product is spoiled.

In some embodiments, the detectable signal is indicating that the food product is spoiled. In some embodiments, lack of detectable signal indicates that the food product is not spoiled.

In the description and claims of the application, the words “include” and “have”, and forms thereof, are not limited to members in a list with which the words may be associated.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

### EXAMPLES

### Example 1: Detection of Food Spoilage with Indicator (Concentration of 50 ppm) Embedded in Wool

As detailed below, permeable substrate made of wool fabrics was dyed with Indigo Carmine, through a straightforward one-step process in slightly acidic solution.

Wool fabric pieces (about 65 cm² and/or about 8 gr each piece) were immersed in Indigo Carmine dye solution having dye concentration within the range of 50-250 ppm, and further containing citric acid-1%. Solution/wool ratio (w/w) was between 4:1 to 5:1. Dying was performed at 85-90° C. for 6-7 hours. At the end of the dying process, the wool pieces were rinsed thoroughly with water and dried at room temperature.

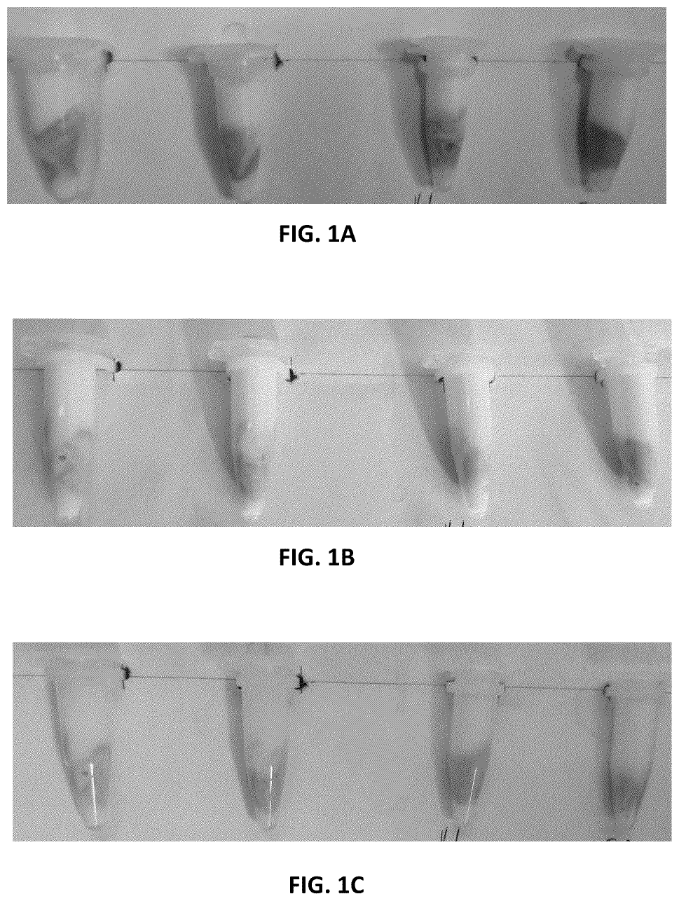

Wool piece dyed in Indigo Carmine dye solution having dye concentration of 50 ppm were immersed within whole milk (about 3.5% fat) contained within vials, a dyed wool piece per vial, and were maintained therein while the vial was sealed (FIG. 1A). The samples were incubated at room temperature (25-27° C.) at t=0, X˜30 hours and Y˜48 hours (FIGS. 1A-C, respectively).

As can be seen, the strong blue color of Indigo Carmine within the fresh milk samples presented in FIG. 1A, gradually faded (FIG. 1B) and eventually after 24-36 hours, the milk sample was spoiled and accordingly the strong blue color disappeared, as shown in FIG. 1C.

### Example 2: Detection of Milk Spoilage Using an Indicator Embedded in Wool

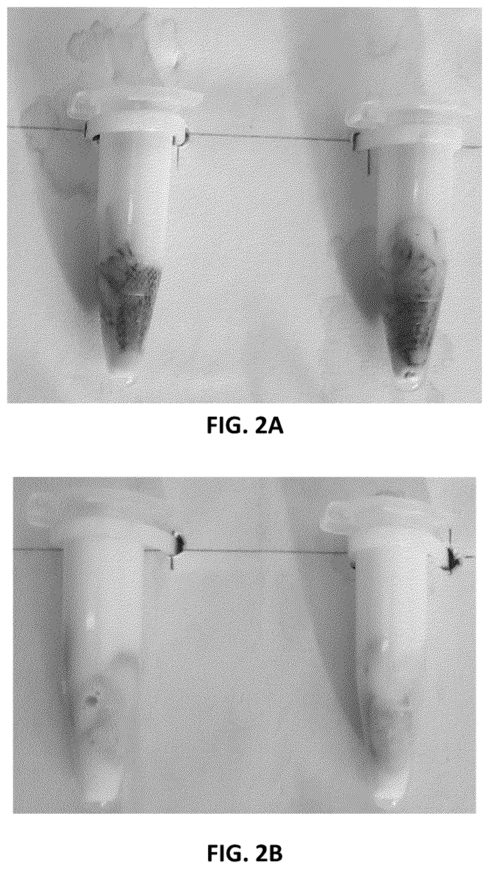

FIGS. 2A and 2B show two fresh samples of milk, each containing a piece of wool dyed with Indigo Carmine solution at a concentration of 60 ppm before and after 36-40 hours incubation in room temperature (25-27° C.), respectively. The milk in FIG. 2B is already spoiled, as reflected by the beige/brown color of the indicators, as opposed to the blue color of the indicator in the fresh samples of milk (FIG. 2A).

### Example 3: Detection of Food Spoilage in Chicken and Red Meet Samples, Using an Indicator Embedded in Cotton

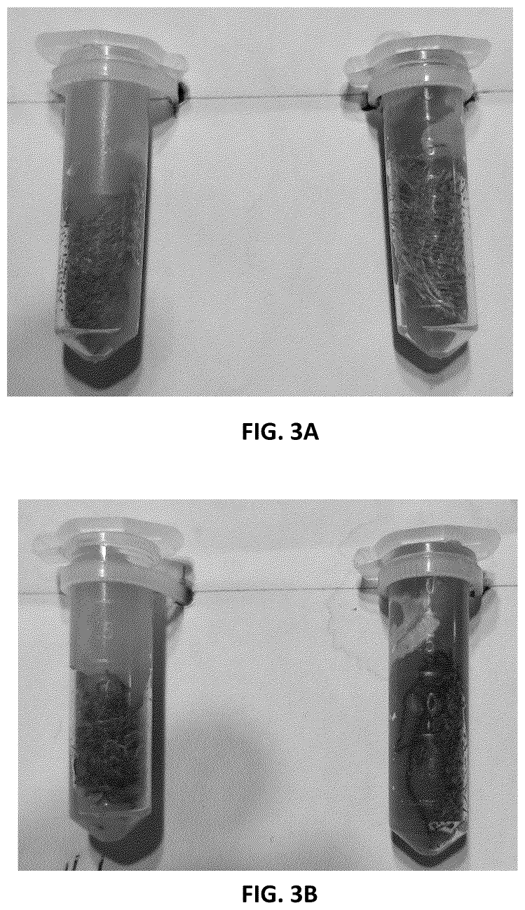

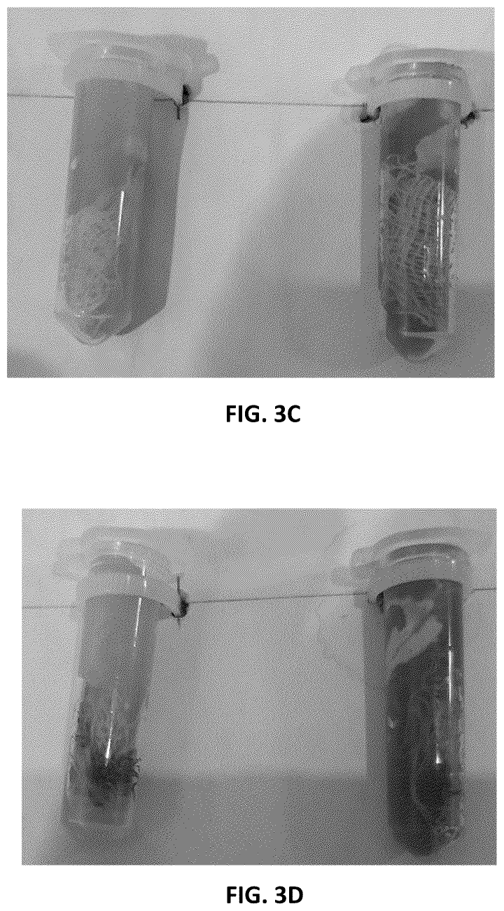

Portions of fresh chicken breast and red meet were wrapped with cotton fabric stained with Indigo Carmine (50-60 ppm in dying solution) pre-treated with neutral cationic solution containing 2,3-epoxypropyltrimethylammonuim chloride as the cationic agent, or pre-treated with basic cationic solution which further contains about 0.036 M KOH (FIGS. 3A and 3B, respectively), left hand panel=chicken; right hand panel=beef.

As shown in FIGS. 3C and 3D (corresponding to FIGS. 3A and 3B, respectively), following rotting of the meet and chicken after 30-36 hours in room temperature, the strong color of the indicator bonded to the cotton fabric decayed.

### Example 4: Detection of Food Spoilage in a Dairy Product Using an Indicator Embedded in Cotton

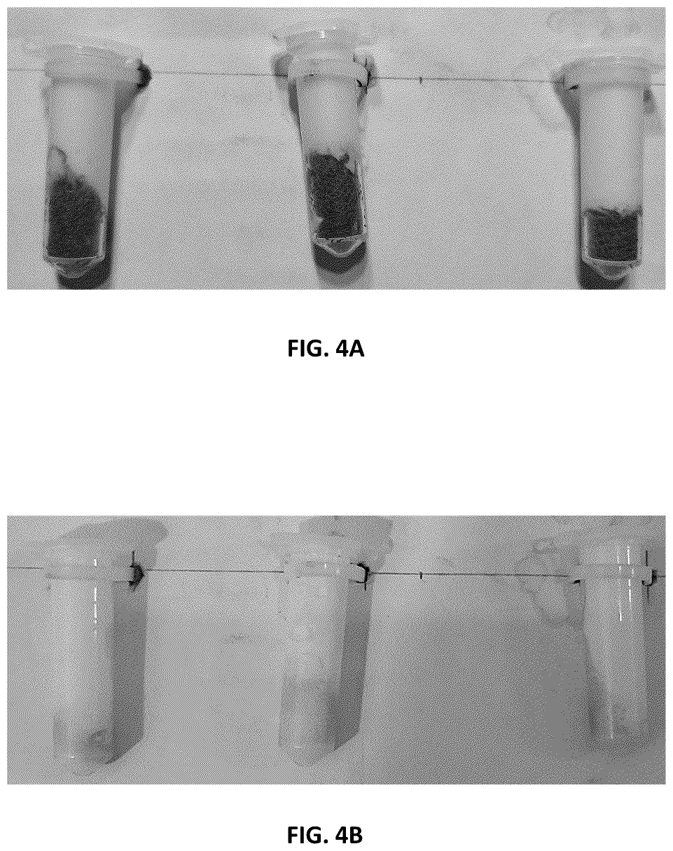

FIGS. 4A and 4B show fresh samples of cottage cheese wrapped in cotton fabric stained with Indigo Carmine dye, after being pre-treated with neutral cationic solution, at t=0 and 28 hours after being incubated in room temperature, respectively.

As shown in FIG. 4B, the strong blue color of the indicator completely disappeared in the spoiled cottage cheese samples.

### Example 5: Detection of Soymilk Spoilage Using an Indicator Embedded in Cotton

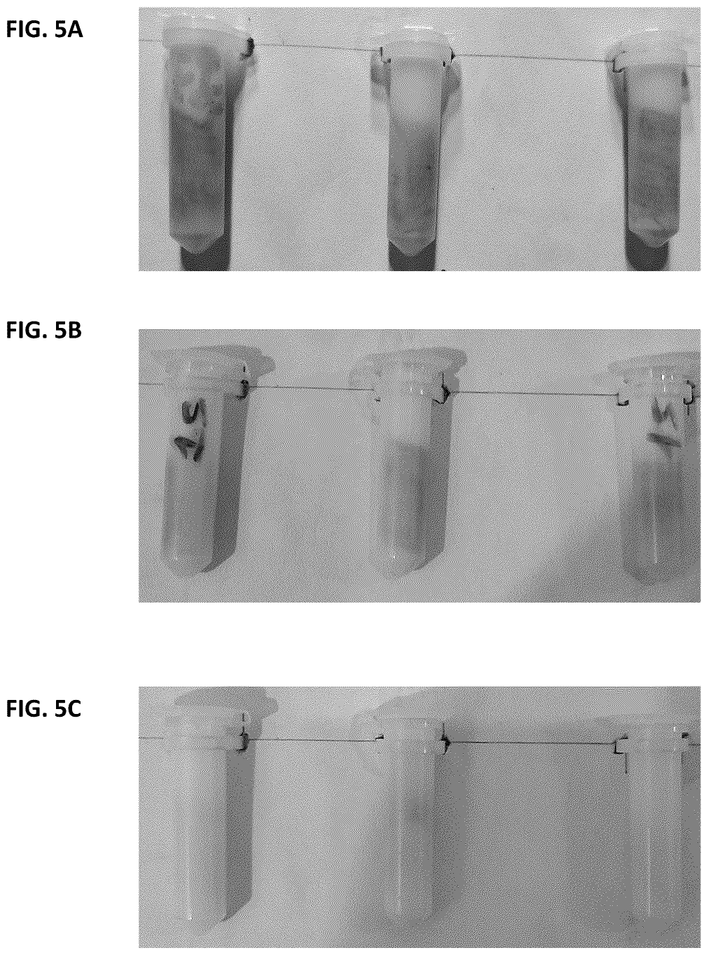

FIGS. 5A to 5C show cotton fabric stained with Indigo Carmine dye, after being pre-treated with neutral cationic solution, immersed within fresh soy milk (FIG. 5A, t=0) and X ˜28-30 hours and Y ˜48 hours of incubation in room temperature (FIGS. 5B and 5C, respectively).

As shown in FIG. 5B, the strong blue color of the indicator decayed in moderately spoiled soymilk to beige/brown after 28-30 hours of incubation in room temperature (FIG. 5B) and completely disappeared in the spoiled soymilk samples after 48 hours of incubation in room temperature (FIG. 5C).

### Example 6: Detection of Food Spoilage in a Diary Product and a Meat Product Using an Indicator Embedded in Cotton

Indicator device (Freshness stickers (FS)) were associated with 5 ml cottage cheese and with 5 ml chicken breast (minced). In addition, indicator device (in the form of patches/stickers) were attached to packaged fresh fish (salmon) meat chunks, and were incubated at room temperature (25° C.) until discoloration was observed.

pH and CFU/ml were measured at the beginning (t0) and at the time point (TP) of discoloration.

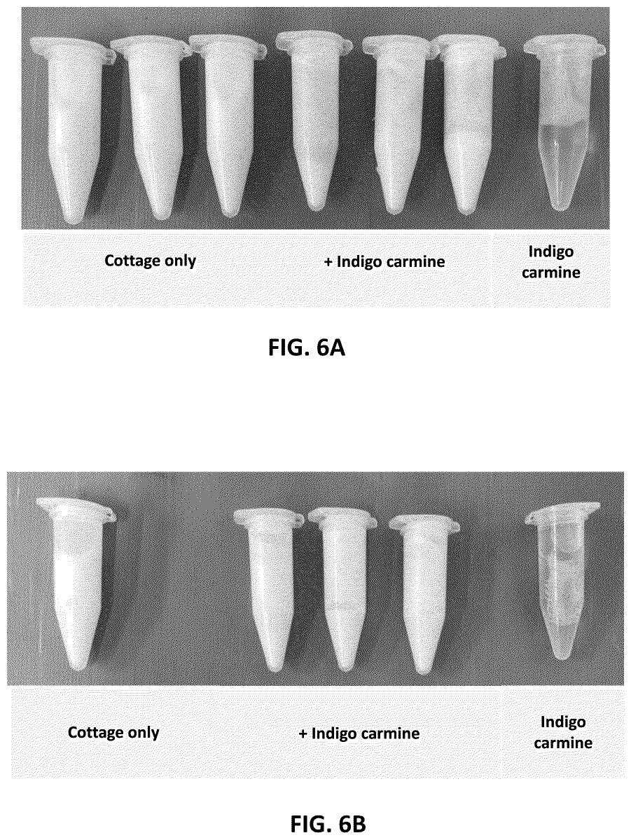

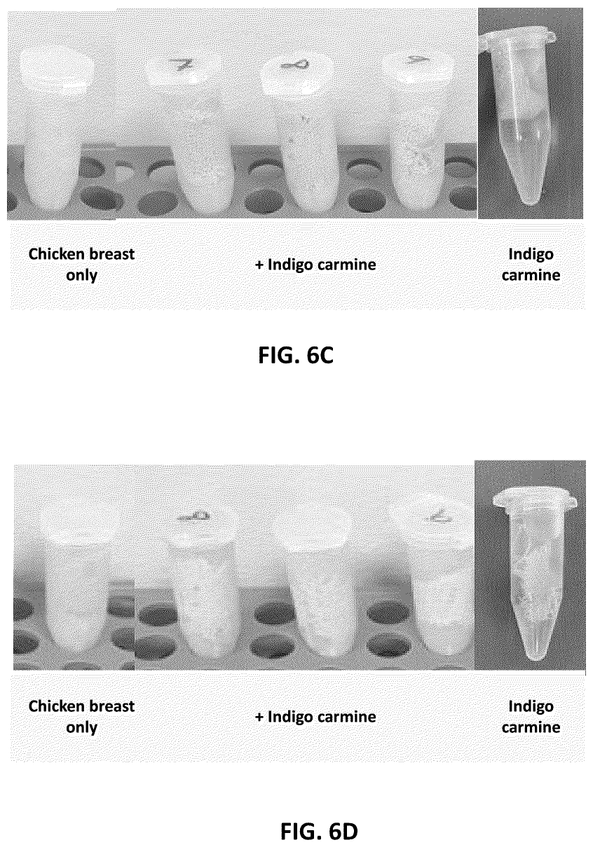

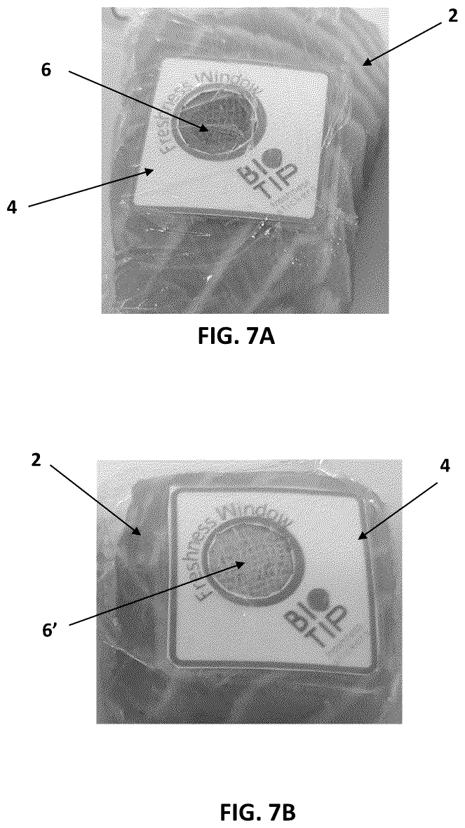

The results are presented in Table 2 below, FIGS. 6A-D and FIGS. 7A-B.

TABLE 2

| | | | | | | CFU/ml | | | Sticker's |
|---|---|---|---|---|---|---|---|---|---|
| | | Initial | pH at TP of | Initial | | at TP of | | TP of | discoloration |
| Product | Indicatior | pH | discoloration | CFU/ml | | discoloration | | discoloration | (%) |
| COTTAGE CHEESE | no (control) | 5.16 | 4.42 | 6.1 × | 10 [symbol] | 1.1 × | 10 [symbol] | 1 day | 100% white |
| COTTAGE CHEESE | yes | 5.16 | 4.37 | 6.1 × | 10 [symbol] | 1.08 × | 10 [symbol] | 1 day | 100% white |
| CHICKEN BREAST | no (control) | 6.06 | 5.99 | 2 × | 10 [symbol] | 4.2 × | 10 [symbol] | 1 day | 100% white |
| CHICKEN BREAST | yes | 6.06 | 6.04 | 2 × | 10 [symbol] | 1.2 × | 10 [symbol] | 1 day | 100% white |

[symbol]

indicates data missing or illegible when filed

The results indicates that in the products, namely, cottage cheese (FIGS. 6A-B), chicken breast (FIGS. 6C-D), as well as the fish meat (FIGS. 7A-B), all the indicator devices changed color from blue to white, one day from experiment initiation.

Reference is made to FIGS. 7A-B, showing packaged fish chunk 2 having indicator device 4 attached thereto. Also shown is indicator 6 (associated with the substrate) changing color from a dark color in the fresh sample (FIG. 7A) to a brighter color (6′) in the spoiled sample (FIG. 7B). The results demonstrate the ability of the indicator device to provide visual indication as to the freshness/spoilage of a food product.

The results thus show that the indicator device can indeed be used for reliable detection of food spoilage and the indicator is inert, hence it does not affect development of spoilage of the food.

### Example 7—Preparation of Indicator Device Having an Additional Coating or Mediator Layer

Preparation of Mediator layer: 100% cotton fabric was treated in aqueous solution composed of tap water, cationic agent (2,3-102Epoxypropylthylammonium chloride) and NaOH and incubated at a temperature in the range of 80-120° C. for 30-180 minutes and dried thereafter.

Substrate and Indicator preparation: 100% cotton fibric was treated an aqueous solution composed of tap water, cationic agent (2,3-102Epoxypropylthylammonium chloride) and NaOH and incubated at a temperature in the range of 80-120° C. for 30-180 minutes and dried thereafter. The treated cotton was then dyed with an indicator composition, including indictor concentration in the range 10 to 100 ppm in aqueous solution. The indicator composition includes one or more types of indicators, for example, 10-100 ppm of indigo carmine, a mixture of 10-50 ppm indigo carmine and 5-50 ppm of tartrazine. The dying procedure performed at a temperature of 85-100° C.

Fabrication of the device: When using a mediator layer-following mediator layer and substrate drying, the substrate and the mediator layer are adhered using an approved food contact glue based on PVA solution and additives such as propylene glycol, lecithin, glycerol, and/or chitosan.

when using coating layer: Following substrate drying, it is coated with a film layer which includes a mixture of PVA, acetic acid and propylene glycol, lecithin, glycerol, and/or chitosan.

Device Performance tests (indicator leakage): all the experiments were performed on products purchased in the various market chains. At experiment initiation, the expiration date was recorded, and the food product was sampled for a general bacterial count. The indicator devices were attached to a slice of the food product, (e.g., chicken breast) and where indicated, were immediately vacuumed. The vacuumed products with the device were kept at fridge temperatures and each day were monitored for decolorization.

At decolorization date, samples were sampled for a total bacteria count and visual indicator leakage on the food product was documented.

Total bacteria count protocol was applied according to the established Israeli and US food administrations standards.

### Example 8—Detection of Microorganism Growth on Fresh or Autoclaved Food Samples Using the Indicator Device

To demonstrate a relation between visual indication of an indicator composition of the indicator device and the presence of microorganisms in a food product, several experiments were performed.

Chicken breast samples were used as food product. First, indicator device including an indicator composition of 20 ppm indigo carmine, was placed on a slice of fresh chicken breast or on a slice of sterile (autoclaved) chicken breast. Samples were kept at fridge temperature (4° C.) and monitored for color change (indicative of food spoilage) and total bacteria count, at the indicator device decolorization time point (1-20 days from initiation of the experiments).

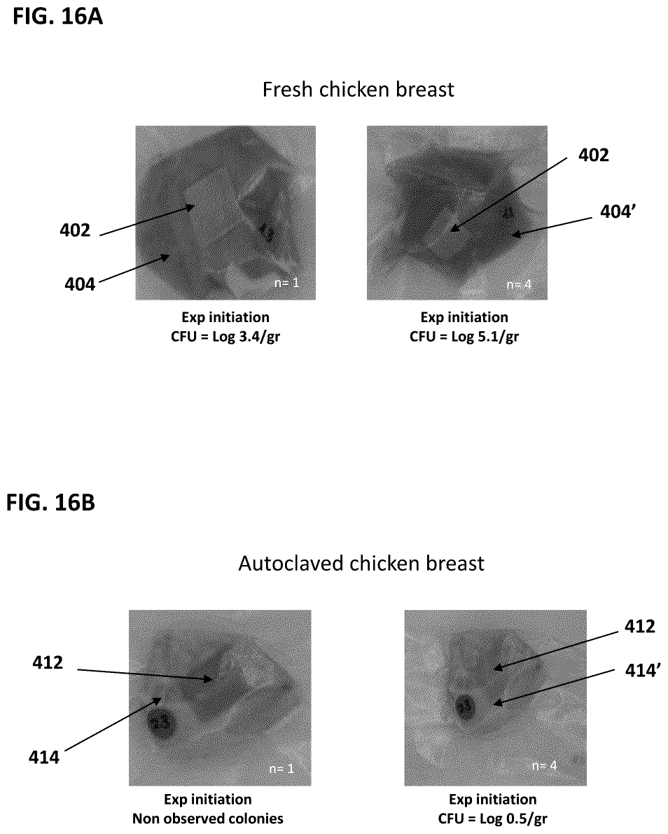

The results are presented in FIGS. 16A-B—The device (402) on the fresh chicken breast (404) changed color on the spoiled chicken breast 404′, at a bacterial level of CFU=Log 5.1/gr (FIG. 16A,), after an incubation period. In contrast, at the same incubation period, the device (412) on the sterilized slices 414, 414′ (FIG. 16B) did not change color and bacterial growth was not detected. It is noted that the autoclave treatment killed all the bacteria on the food product 414, 414′ (FIG. 16B).

The results indicate that the indeed, the color indication (in this example, discoloration) of the indicator composition of the indicator device occurs due to the presence and development of microbes on the food product.

### Example 9—Determination of Indicator Device Placement Location on a Met Food Product

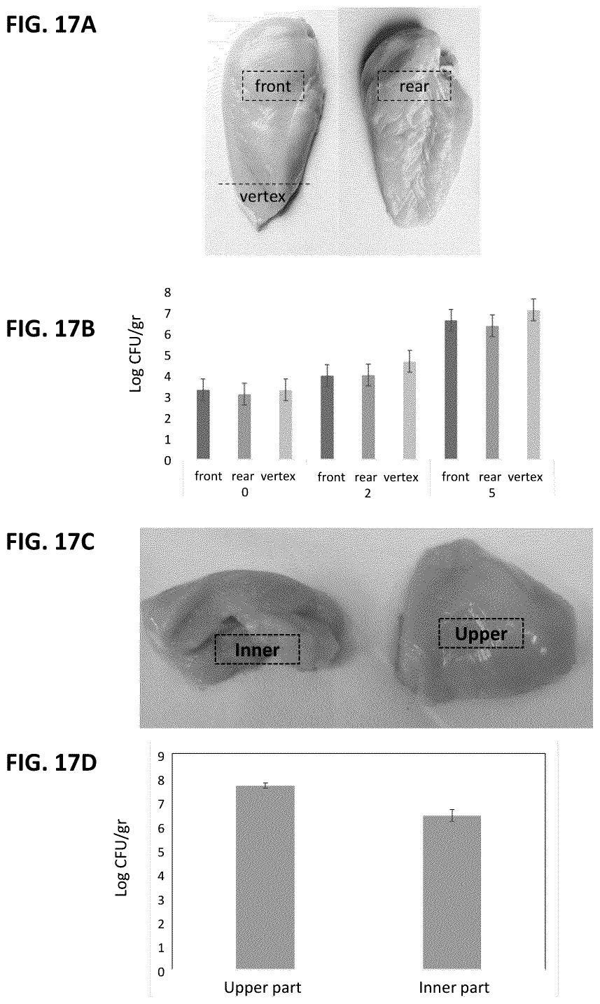

In order to determine whether there is a representative area in a chicken breast food product for placement of an indicator device, the levels of bacteria in different areas were examined (rear region vs front vs vertex). Total bacteria count was measure at purchasing day (0) and 2 and days from experimental initiation. All samples were kept at fridge temperatures along the whole experiment. The results presented in FIGS. 17A-B indicate that no significant differences were identified in the levels of bacteria at the beginning and throughout the experiment whether the front, vertical or the rear parts were sampled.

Next, the difference in the levels of bacteria on the outside of the slice versus the inside of the slice were tested. The results are presented in FIG. 17C-D. In chicken breast that has been kept at refrigerator temperature for several days, it was found that the levels of bacteria on the outside (“Upper” portion) are significantly higher than on the inside (“Inner” portion). External part CFU=Log 7.7/gr vs. inner part CFU=Log 6.4/gr.

Collectively, these experiments indicate that the most representative area for the placement of the indicator device is the outer part of the slice from the front, since this area represents the maximum amount of bacteria to which the indicator composition of the device can react and it is also the most visible area from a consumer perspective.

### Example 10—Detection of Food Spoilage Using an Indicator Device at Various Spoilage Levels

Indicator devices including various indicator compositions were tested for their ability to provide indication regarding food spoilage at different levels of spoilage (i.e., at different levels of microorganisms) at various forms of food packaging.

I—Indicator composition including 10 ppm indigo carmine. The indicator device was placed on a simple food packaging (polyethylene wrap), to determine CFU at discoloration, and was placed with a vacuum pack to determine CFU at discoloration.

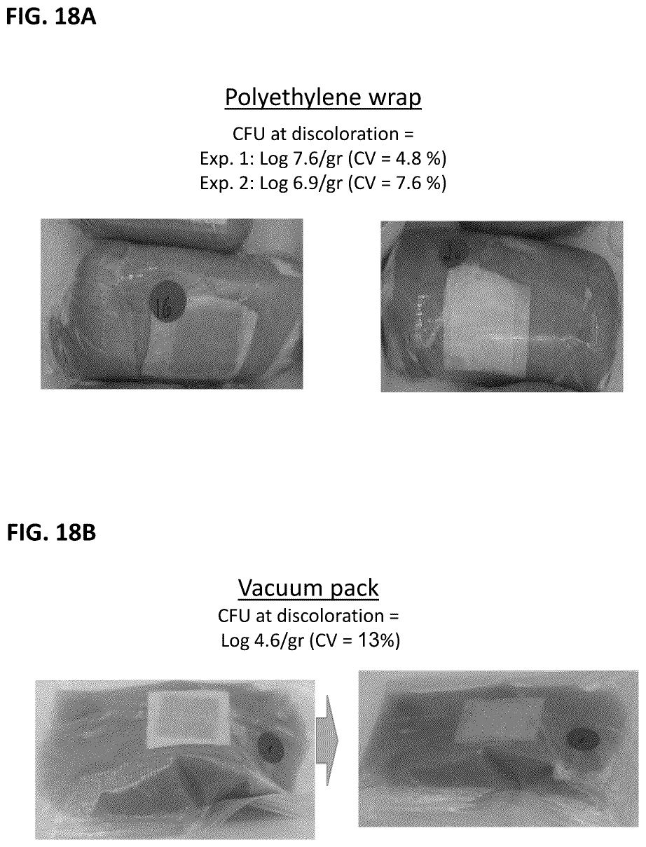

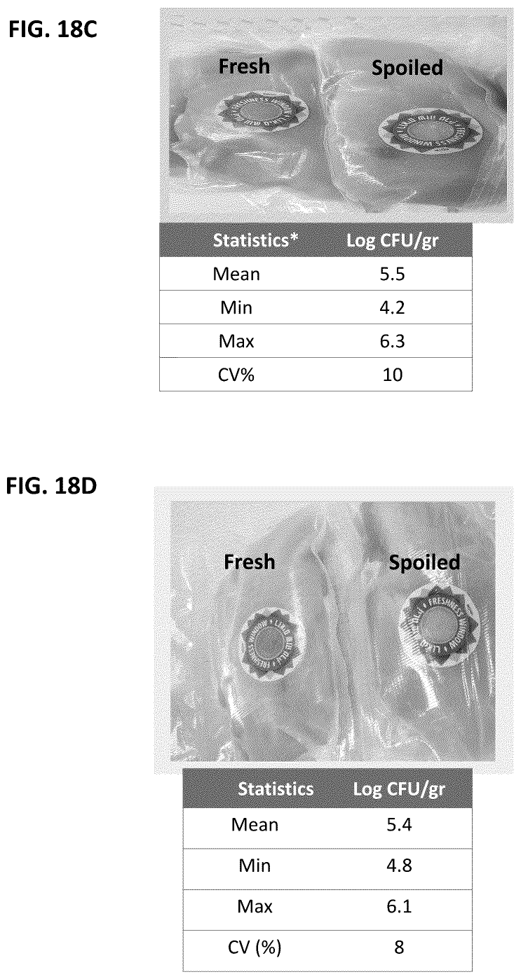

The device was calibrated to decolorate at spoilage level for use with vacuum packaging. In this example, the device was prepared as described in Example 7, (including a mediator layer and excluding a top cover). The results are presented in FIGS. 18A-C. As shown, the indicator device changes color at the defined spoilage level while using vacuum packaging (FIG. 18B, 18C). The color change occurs at high values of CFU, and the meat is actually spoiled when a simple polyethylene wrap is used (FIG. 18A, results of two experiments (Exp. 1 and Exp. 2) are shown).

Additionally, in order to improve the uniformity of the discoloration time point, a top cover may be used, to reduce the coefficient of variation (CV), for example, from CV=13% to CV=7.7% with an average count of Log CFU=5.4/gr. The type of the top cover can reduce the CV (the variations among the tested samples). The sample which exhibited lower variation (CV=7.7%), is an approved food contact top cover

The results indicate that discoloration indeed occurs at a defined spoilage levels with relatively high uniformity.

II—Indicator composition including 10 ppm indigo carmine and 5 ppm tetrazine:

The device was prepared as above. The experiment was conducted at fridge temperature and all breast slices were packaged in vacuum. The results are presented in FIG. 18D and show that the colour change point occurs at Log CFU values=5.4 with CV=8%. These results are with agreement with the former ones, showing high uniformity and repeatability.

III—Indicator composition including 15 ppm indigo carmine:

In this example, the device was prepared as described in Example 7, (including a coating layer. The device was tested on chicken breast using vacuum packaging. The experiment was conducted at fridge temperature. In this experiment it was found that the discoloration time point occured at average of 1.25 days from product expiration date, similarly to results obtained for indicator devices including a mediator layer (as in experiments I and II). Furthermore, the results indicated that no leakage was observed with any of the indicator devices.

Collectively, the results indicate that discoloration occurs at a defined spoilage levels with high uniformity and repeatability, and without detectable levels of indicator leakage to the food sample. The results further demonstrate and substantiate the advantageous properties of the indicator device of providing safe, reliable and accurate indication regarding food spoilage.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

1.-52. (canceled)

53. An indicator device for detecting food spoilage of a food product, the indicator device comprising:
a. a substrate; and
b. an indicator composition comprising at least one indicator, the indicator composition is associated with the substrate,
wherein said substrate is configured to be in contact with the food product, said indicator composition is configured to produce a detectable signal upon reacting with at least a portion of the food product, or of components secreted therefrom.

54. The indicator device according to claim 53, wherein said substrate comprises textile fibers, plastic, nylon, woven fibers, woven fabrics, non-woven fabrics, artificial polymers, mixed fibers, biodegradable polymers, cotton, wool, linen, oligo-sugars, polysaccharides, gels, modified starch, metals, aluminum, paper, carton, wood, silk, plant material, clay, adhesives, ointment, vax, oil, leather, carbon and graphite felt, carbon fibers, or any combination thereof.

55. The indicator device according to claim 53, wherein the substrate further comprises one or more additional layers.

56. The indicator device according to claim 55, wherein the additional layer comprises or is made of textile fibers, plastic, nylon, woven fibers, non-woven fibers, cotton, wool, silk, cellulose-starch chitosan-PVA, cellulose-PVA cross linked film, or any combination thereof.

57. The indicator device according to claim 55, wherein the additional layer is permanently or transiently associated with the substrate.

58. The indicator device according to claim 55, wherein the additional layer is stitched, adhered and/or welded to the substrate.

59. The indicator device according to claim 53, wherein said spoilage is microbial related spoilage.

60. The indicator device according to claim 53, wherein said at least one indicator is selected from microbial indicators, azo dye, food colors, natural food dyes, Thiazine dye, redox indicators, pH indicators, oxygen indicator, solvatochromic dye, or any combination thereof.

61. The indicator device according to claim 53, wherein the at least one indicator is provided in a concentration such that it changes at least one detectable property in response to spoilage of the food sample, said property comprises spectroscopic, colorimetric, fluorometric and/or electrochemical property.

62. The indicator device according to claim 53, wherein the at least one indicator is provided in a concentration such that it changes its color in response to spoilage of the food sample.

63. The indicator device according to claim 53, wherein at least one indicator is a pH indicator and wherein the indicator is configured to change color upon a change of pH smaller than about 0.5 pH unit.

64. The indicator device according to claim 53, wherein the at least one indicator is a bacterial indicator configured to provide a detectable indication in the presence of a bacterial population above a pre-determined threshold, presence of secreted volatile compounds above a threshold, presence of specific types of bacterial population, or any combination thereof.

65. The indicator device according to claim 53, wherein the indicator composition comprises a plurality of indicators.

66. The indicator device according to claim 53, wherein the substrate is associated with a plurality of indicator compositions.

67. The indicator device according to claim 53, wherein the indicator device is or is comprised with a food package configured to at least partially hold or wrap the food.

68. A method for detecting food spoilage of a food product, the method comprising the steps of:
placing the indicator device of claim 53, in a contact with a food product;
allowing at least a food sample and/or secreted materials of said food product to contact said indicator; and
determining whether said food product is spoiled upon detecting a detectable signal generated by said indicator, if or when the food sample is spoiled.

69. A system for detecting food spoilage of a food product, the system comprising:
at least one food packaging configured to hold or wrap a food product; and
an indicator device according to claim 53, associated with the food packaging.

70. The system according to claim 69, wherein the food product is selected from fluid food product, liquid food product, soft food product, semi-hard food product, solid, hard food product and aqueous food product.

71. The system according to claim 69, wherein the food packaging is selected from a container, a vial, a box, a tray, a can and a palette, a bag, a wrap, or any combinations thereof.

72. The system according to claim 69, wherein the indicator device is integrally formed with the food packaging, or is permanently or transiently associated with the food packaging.
